(19) United States
(12) Patent Application Publication
(10) Pub. No.: US 2023/0273210 A1
(43) Pub. Date: Aug. 31, 2023

(54) SYSTEMS AND METHODS FOR PREDICTING GRAFT DYSFUNCTION WITH EXOSOME PROTEINS

(71) Applicant: THE TRUSTEES OF COLUMBIA UNIVERSITY IN THE CITY OF NEW YORK, New York, NY (US)

(72) Inventors: Barry Fine, Mamaroneck, NY (US); Nicholas Tatonetti, New York, NY (US); Nicholas Giangreco, Buffalo, NY (US)

(73) Assignee: THE TRUSTEES OF COLUMBIA UNIVERSITY IN THE CITY OF NEW YORK, New York, NY (US)

(21) Appl. No.: 18/180,991

(22) Filed: Mar. 9, 2023

Related U.S. Application Data
(63) Continuation of application No. PCT/US21/50465, filed on Sep. 15, 2021.
(60) Provisional application No. 63/436,978, filed on Jan. 4, 2023.
(60) Provisional application No. 63/078,672, filed on Sep. 15, 2020.

(51) Int. Cl. G01N 33/573; G06N 3/08; G06N 7/01; G06N 20/00

(52) U.S. Cl. CPC G01N 33/573; G06N 3/08; G06N 7/01; G06N 20/00

(57) ABSTRACT

Described here are techniques for identifying risk of primary graft dysfunction (PGD) of a subject. The disclosed method can include collecting serum of the subject, measuring a level of a PGD marker from the serum, wherein the PGD marker comprises plasma kallikrein (KLKB1), providing a PGD risk value that is quantified based on the level of the PGD marker using an adaptive Monte Carlo cross-validation (MCCV) model, and identifying the risk of PGD based on the PGD risk value.

### CROSS-REFERENCE TO RELATED APPLICATION

This application claims priority to U.S. Provisional Patent Application No. 63/436,978, filed on Jan. 4, 2023, and PCT Application No. PCT/US21/50465, filed on Sep. 15, 2021, which claims the benefit of U.S. Provisional Patent Application No. 63/078,672, filed on Sep. 15, 2020, the entire content of each of which are incorporated by reference herein.

### GRANT INFORMATION

This invention was made with government support under grant number UL1 TR001873 and K08HL140201 awarded by the National Institutes of Health. The government has certain rights in the invention.

### BACKGROUND

Heart transplantation is a recognized treatment option for patients with end stage heart failure. As organ availability is limited, it can be important to carefully assess the risk of transplant candidates to improve transplant outcomes and organ allocation.

Primary graft dysfunction (PGD) after heart transplant can be defined as idiopathic heart failure occurring within the immediate postoperative period. PGD can affect either or both ventricles simultaneously and be graded from mild to severe depending on the amount of support required to compensate for organ dysfunction. PGD can cause the death of patients within 30 days after transplant.

The underlying cause of PGD and the importance of different factors towards post-transplant PGD remains unclear. Identifying predictive factors of PGD in recipients has the potential to improve risk stratification, organ allocation, and post-operative care as well as increase the understanding behind the etiology of PGD.

Improving the prediction of post-transplant survival can improve the use of available grafts and to better assess the risks and benefits of transplantation for high-risk patients. Tools that can accurately classify post-transplant risk have been developed for other solid organ transplants, such as kidney and lung, but similar efforts to predict post-transplant survival in heart transplantation have had limited success.

Circulating microvesicles are small vesicles that contain proteins, RNA, and DNA and play a role in intercellular communication throughout the body. The proteome of microvesicles, which can be purified and analyzed using mass spectrometry, has been shown to be a valuable resource for identifying novel biomarkers. Certain studies demonstrated the utility of microvesicle proteomics for predicting primary graft dysfunction before transplant and for diagnosing cellular and antibody-mediated rejection.

As such, there is a need in the art for improved techniques for predicting PGD, and techniques to overcome certain challenges due to the limitations of poor discrimination in external validation set and to outperform the current methods by expanding the pool of potential transplant biomarkers associated with transplant survival using macrovesicle proteomics.

### SUMMARY

The disclosed subject matter provides techniques for identifying the risk of primary graft dysfunction (PGD) of a subject.

An exemplary method can include collecting a sample of the subject, measuring a level of a PGD marker from the sample, providing a PGD risk value that is quantified based on the level of the PGD marker using an adaptive Monte Carlo cross-validation (MCCV) model, and identifying the risk of PGD based on the PGD risk value. In non-limiting embodiments, the PGD marker can include plasma kallikrein (KLKB1).

In certain embodiments, the method can further include assessing an effect of a therapy on the heart transplant by estimating the PGD risk value of the subject. The subject can receive the therapy before or after the assessment.

In certain embodiments, the method can further include identifying a clinical variable of the subject. In non-limiting embodiments, the clinical variable can include a medical history of the subject. In some embodiments, the medical history of the one subject can include a pre-transplant inotrope therapy.

In certain embodiments, the method can further include measuring a level of an additional marker from the sample. In non-limiting embodiments, the additional marker can include proteins peroxiredoxin 2 (PRDX2), tropomyosin alpha-4 (TPM4), myeloperoxidase (MPO), PGLYRP2, DEFA1, DEFA1B, LDHB, F2, FCGBP, CAT, CFHR5, HIST1H4, GAPDH, LTF, ADIPOQ, HSPA5, or combinations thereof.

In certain embodiments, the PGD risk value can be quantified based on the level of the PGD marker and the additional marker.

In certain embodiments, the method can further include providing the adaptive MCCV model with a training set for machine learning. In non-limiting embodiments, the adaptive MCCV model can be a continuously evolving model based on the training set.

In certain embodiments, the method can further include providing an additional therapy to the subject based on the PGD risk value. In non-limiting embodiments, the additional therapy can include KLKB1 activators, anti-inflammatory agents, or combinations thereof.

The disclosed subject matter also provides methods for predicting post-transplant survival of a subject seeking an organ transplant.

An exemplary method can include collecting a sample from the subject, measuring in the sample, a level of a marker predictive of post-transplant survival, providing a transplant risk value that is quantified based on the level of the marker using an adaptive Monte Carlo cross-validation (MCCV) model, and predicting the likelihood of post-transplant survival based on the transplant risk value.

In non-limiting embodiments, the marker predictive of post-transplant survival is at least one of prothrombin (F2), anti-plasmin (SERPINF2), Factor IX (F9), carboxypeptidase 2 (CPB2), HGF activator (HGFAC) and low molecular weight kininogen (LK). In some embodiments, a level of F2, SERPINF2, F9, CPB2, or HGFAC, outside a distribution of values in a survival cohort or a level of LK outside a distribution of values in a survival cohort predicts post-transplant survival of the subject.

In some embodiments, predicting post-transplant survival identifies a risk of primary graft dysfunction (PGD)

In certain embodiments, the method can further include providing the adaptive MCCV model with a training set for machine learning. In non-limiting embodiments, the adaptive MCCV model can be a continuously evolving model based on the training set.

In certain embodiments, the method can further include providing a therapy to the subject based on the transplant risk value. In non-limiting embodiments, the therapy can provided before or after the organ transplant.

In certain embodiments, the method can further include identifying a clinical variable of the subject. In non-limiting embodiments, the clinical variable can include a medical history of the subject.

The disclosed subject matter further provides systems for identifying the risk of primary graft dysfunction (PGD) of a subject. An example system can include one or more processors and one or more computer-readable non-transitory storage media coupled to one or more of the processors. The one or more computer-readable non-transitory storage media can include instructions operable when executed by one or more of the processors to cause the system to collect a sample of the subject, measure a level of a PGD marker from the sample, provide a PGD risk value that is quantified based on the level of the PGD marker using an adaptive Monte Carlo cross-validation (MCCV) model, and identify the risk of PGD based on the PGD risk value. In non-limiting embodiments, the PGD marker can include plasma kallikrein (KLKB1).

The disclosed subject matter further provides systems for predicting post-transplant survival of a subject seeking an organ transplant. An exemplary system can include one or more processors and one or more computer-readable non-transitory storage media coupled to one or more of the processors. The one or more computer-readable non-transitory storage media can include instructions operable when executed by one or more of the processors to cause the system to collect a sample from the subject, measure in the sample, a level of a marker predictive of post-transplant survival, provide a transplant risk value that is quantified based on the level of the marker using an adaptive Monte Carlo cross-validation (MCCV) model, and predict the likelihood of post-transplant survival based on the transplant risk value.

In non-limiting embodiments, the marker predictive of post-transplant survival is at least one of prothrombin (F2), anti-plasmin (SERPINF2), Factor IX (F9), carboxypeptidase 2 (CPB2), HGF activator (HGFAC) and low molecular weight kininogen (LK). In some embodiments, a level of F2, SERPINF2, F9, CPB2, or HGFAC, outside a distribution of values in a survival cohort, or a level of LK outside a distribution of values in a survival cohort predicts post-transplant survival of the subject.

In certain embodiments, the processor is configured to identify a clinical variable of the subject. In non-limiting embodiments, the clinical variable can include a medical history of the subject. In some embodiments, the medical history of the one subject can include a pre-transplant inotrope therapy.

In certain embodiments, the processor is configured to provide the adaptive MCCV model with a training set for machine learning. In non-limiting embodiments, the adaptive MCCV model can be a continuously evolving model based on the training set.

The disclosed subject matter will be further described below.

### BRIEF DESCRIPTION OF THE DRAWINGS

FIG. 1A provides a diagram of example blood-derived micro-vesicle proteomics in accordance with the disclosed subject matter. FIG. 1B provides a diagram showing an example protein markers identified by mass spectrometry in accordance with the disclosed subject matter. FIG. 1C provides example protein filtering in accordance with the disclosed subject matter.

FIG. 2 provides a graph showing clinical diagnostic ELISA tests for C3, C4, total complement proteins in accordance with the disclosed subject matter.

FIG. 3 provides a diagram showing Monte Carlo Cross-Validation (MCCV) Prediction in accordance with the disclosed subject matter.

FIG. 4 provides a graph showing exosome protein expression distributions for patient cohorts in accordance with the disclosed subject matter.

FIG. 5 provides a graph showing example techniques for primary graft dysfunction (PGD) prediction by clinical and protein markers in accordance with the disclosed subject matter.

FIG. 6 provides a graph showing the prediction of pre-transplant inotrope therapy, left ventricular assist device, and both clinical factors on posttransplant PGD in accordance with the disclosed subject matter.

FIG. 7A provides a graph showing the area under the receiver operating characteristic curve (AUROC) in accordance with the disclosed subject matter. FIG. 7B provides a graph showing the AUROC distribution for all panels per marker composition in accordance with the disclosed subject matter. FIG. 7C provides a graph showing the AUROC distribution for all marker panels composed of at least 1 protein marker and all inotrope therapy panels in accordance with the disclosed subject matter.

FIG. 7D provides a graph showing the AUROC performance of 2 marker panels comparison overall against the average of individual cohorts and the integrated cohort in accordance with the disclosed subject matter. FIG. 7E provides a graph showing the performance vs. the variation of the performance between the three patient cohorts in accordance with the disclosed subject matter. FIG. 7F provides the KLKB1 and inotrope therapy PGD classifier equation in accordance with the disclosed subject matter.

FIG. 8 provides graphs showing pre-transplant KLKB1 protein expression and inotrope therapy predict post-transplant PGD in accordance with the disclosed subject matter.

FIG. 9 provides graphs showing clinical and protein panel that outperforms existing clinical predictors in accordance with the disclosed subject matter.

FIG. 10A provides a graph showing a normalized ELISA KLKB1 concentration comparison in accordance with the disclosed subject matter. FIG. 10B provides a graph showing the putative PGD classifier in accordance with the disclosed subject matter.

FIG. 10C provides example performance metrics of the classifier at the highest sensitivity in accordance with the disclosed subject matter.

FIG. 11 provides a diagram showing a differential protein analysis modeling scheme in accordance with the disclosed subject matter.

FIG. 12A provides a graph showing enrichment and depletion of pathways using differential protein expression in accordance with the disclosed subject matter. FIG. 12B provides a graph showing protein marker predictors in accordance with the disclosed subject matter. FIG. 12C provides a graph showing ESR expression in accordance with the disclosed subject matter. FIG. 12D provides a graph showing hsCRP expression in accordance with the disclosed subject matter.

FIG. 13 provides a graph showing a calibration curve for PGD prediction by a putative classifier on 80 CUIMC patient assessment data in accordance with the disclosed subject matter.

FIG. 14A provides a graph showing overlay of protein expression and association for site-of-origin covariates for patients via Principal Components Analysis. FIG. 14B provides a graph showing overlay of protein expression and association for Set covariates for patients via Principal Components Analysis. FIG. 14C provides a graph showing overlay of protein expression and association for TMT-Tag covariates for patients via Principal Components Analysis.

FIG. 15A provides a graph showing a correlation between unadjusted and adjusted panel performances for one marker panels. FIG. 15B provides a graph showing a correlation between unadjusted and adjusted panel performances for two marker panel performances.

FIG. 16 provides an overview of the study described in Example 2.

FIG. 17 provides an exemplary time to patient mortality post-heart transplant.

FIG. 18 provides a graph showing example techniques for patient survival prediction after transplant using clinical and protein markers in accordance with the disclosed subject matter.

FIGS. 19A-19F provide predictive protein distributions and performance for post-transplant survival. FIGS. 19A and 19D representative maximum-minimum normalized protein distributions for patients and replicate samples grouped by patients who survived or died after heart transplant. FIGS. 19B and 19E show representative receiver operating characteristic (ROC) curve between sensitivity and 1-specificity. FIGS. 19C and 19F show representative precision-recall curves.

FIG. 20 provides correlation between prediction of PGD and survival for protein markers.

It is to be understood that both the foregoing general description and the following detailed description are exemplary and are intended to provide further explanation of the disclosed subject matter.

### DETAILED DESCRIPTION

The disclosed subject matter provides techniques for treating and/or preventing primary graft dysfunction (PGD) by analyzing exosome proteins. The disclosed subject matter provides systems and methods for predicting PGD with exosome proteins and treating PGD based on the prediction. The terms primary graft dysfunction (PGD) and primary graft failure (PGF) can be used interchangeably herein.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold, of a value.

The term “coupled,” as used herein, refers to the connection of a device component to another device component by methods known in the art.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc.

In certain embodiments, the disclosed subject matter provides a method for identifying the risk of primary graft dysfunction (PGD) of a subject. An example method can include collecting a sample of the subject, measuring a level of PGD marker from the sample, providing a PGD risk value, and identifying the risk of PGD based on the PGD risk value.

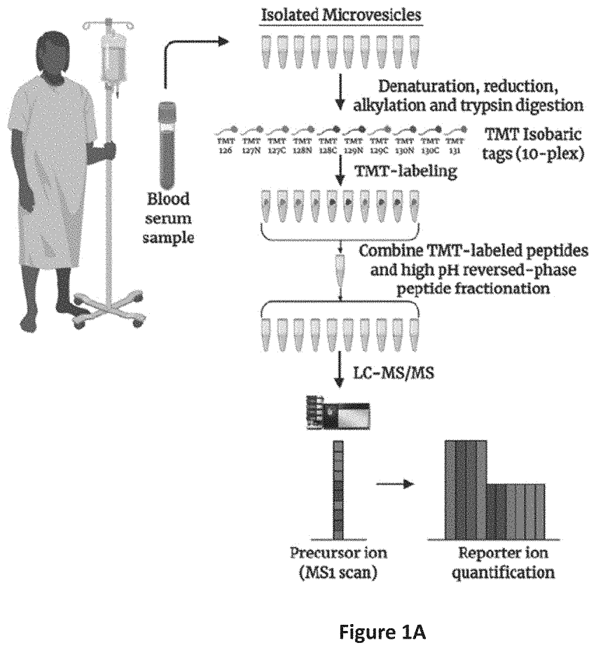

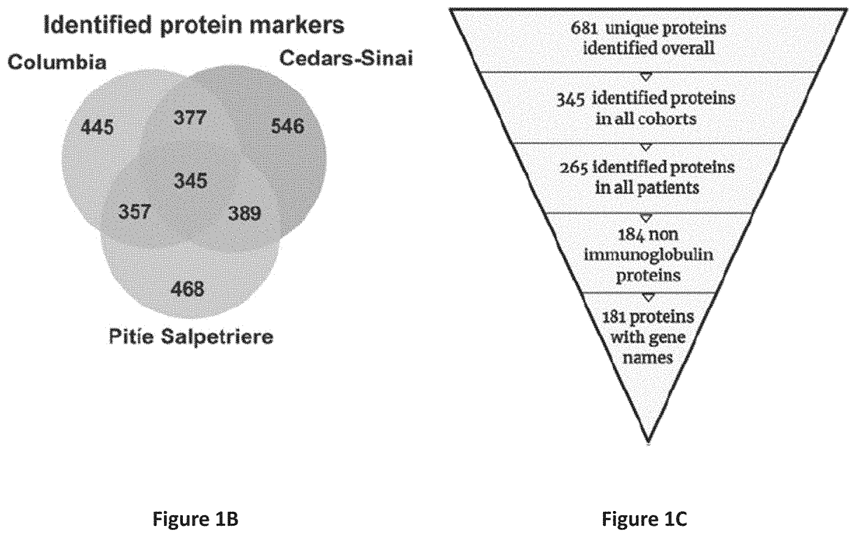

In certain embodiments, as shown in FIG. 1, the sample can be collected from a subject. In non-limiting embodiments, the sample can include any body fluids of the subject. For example, the sample can include blood, serum, tears, effluent fluids, plasma, urine, semen, saliva, bronchial fluid, cerebral spinal fluid (CSF), amniotic fluid, synovial fluid, lymph, bile, gastric acid, or combinations thereof.

In certain embodiments, the method can include obtaining one or more characteristics of the subject. The characteristic can include demographics, biometrics, lab values, medications, hemodynamics, cardiomyopathy, transplant factors, clinical variables or combinations thereof. For example, the demographics can include body mass index (BMI), blood type, age, sex, history of tobacco, diabetes, ischemic, or combinations thereof. The cardiomyopathy can include non-ischemic, Adriamycin, amyloid, Chagas, Congenital, Hypertrophic cardiomyopathy, Idiopathic, Myocarditis, Valvular Heart Disease, Viral, Ischemic Time, or combination thereof. The transplant factors can include ventricular assist device, pulmonary artery (PA) diastolic, or a combination thereof. The hemodynamics can include pulmonary artery systolic, PA mean, central venous pressure (CVP), pulmonary capillary wedge pressure (PCWP), creatinine, or a combination thereof. The lab values can include an international normalized ratio (INR), total bilirubin, sodium, antiarrhythmic, or combinations thereof. The medications can include beta-blocker, inotrope, CVP/PCWP, or combinations thereof. The clinical variables can include a medical history of the subject (e.g., pre-transplant inotrope therapy). In non-limiting embodiments, the characteristic can be used for calculating radial score (RADIAL) and model for end-stage liver disease score (MELD) scores. For example, the MELD score can be derived for each patient using the formula:

3.78×ln[serum bilirubin (mg/dL)]+11.2×ln[INR]+9.57×ln[serum creatinine (mg/dL)]+6.43 (1)

In non-limiting embodiments, clinical risk scores can include a plurality of risk factors for primary graft dysfunction (e.g., Right atrial pressure >=10 mm Hg, recipient Age>=60 years, Diabetes mellitus, Inotrope dependence, donor Age>=30 years, Length of ischemic time>=240 minutes—i.e., RADIAL score).

In certain embodiments, the level of a PGD marker can be measured from the sample of the subject. In non-limiting embodiments, the PGD marker can include proteins peroxiredoxin 2 (PRDX2), tropomyosin alpha-4 (TPM4), myeloperoxidase (MPO), PGLYRP2, DEFA1, DEFA1B, LDHB, F2, FCGBP, CAT, CFHR5, HIST1H4, GAPDH, LTF, ADIPOQ, HSPA5, plasma kallikrein (KLKB1), or combinations thereof. In non-limiting embodiments, the PGD marker can be KLKB1. In some embodiments, the method can further include measuring the level of the additional marker from the sample. The additional marker can include PRDX2, TPM4, MPO, PGLYRP2, DEFA1, DEFA1B, LDHB, F2, FCGBP, CAT, CFHR5, HIST1H4, GAPDH, LTF, ADIPOQ, HSPA5, KLKB1, IGHD, IGLV2-11, or combinations thereof.

In certain embodiments, the level of the PGD marker and/or additional maker can be measured through various assays. In non-limiting embodiments, the level of the PGD marker and/or additional maker can be measured using mass spectrometry analysis. For example, microvesicles can be isolated from a sample (e.g., 100 ul) from a subject and homogenized using an MS-compatible lysis buffer. Lysate (e.g., 20 μg) from each sample can be proteolytically cleaved with trypsin and chemically labeled with mass spectrometer detectable quantification reagent. A reference sample can be generated by pooling equal amounts of microvesicles from each subject to create a protein library for quantification. Samples can be bulk mixed (e.g., at 1:1) across all channels, and bulk mixed samples can be fractionated, and each fraction can be dried. Dried peptides can be dissolved in a solution of 2% acetonitrile/2% formic acid and injected (e.g., in Oribitrap Fusion coupled with the UltiMate™ 3000 RSLCnano system). Fractionated peptides can be separated with an about 5-30% acetonitrile gradient in about 0.1% formic acid over about 70 min. In non-limiting embodiments, the full MS spectra were acquired at a resolution of about 120,000. In some embodiments, the method can include selecting the most intense ions (e.g., MS1 ions) for MS2 analysis. MS1 can be the initial ionized sample. These ions can split into smaller fragments usually through collision to generate smaller ions (MS2) and so on (MS3). Each MS represents a greater fragmentation such that the their separation by mass/charge ratio allows to identify individual ions. The isolation width can be set at about 0.7 Da, and isolated precursors can be fragmented by Collision Induced Dissociation (CID) at normalized collision energy (NCE) of 35% and analyzed in the ion trap using “turbo” scan speed. Following the acquisition of each MS2 spectrum, a synchronous precursor selection (SPS) MS3 scan can be collected on the selected ions (e.g., the top 10 most intense ions in the MS2 spectrum). SPS-MS3 precursors can be fragmented by higher energy collision-induced dissociation (HCD) at an normalized collision energy (NCE) of 60% and analyzed. Raw mass spectrometric data can be analyzed using to perform database search and tandem mass tags (TMT) reporter ions quantification. TMT can be isobaric mass tags that can allow for quantitation of each protein identified in mass spec. TMT tags on lysine residues and peptide N termini (e.g., +229.163 Da) and the carbamidomethylating of cysteine residues (e.g., +57.021 Da) can be set as static modifications, while the oxidation of methionine residues (e.g., +15.995 Da), deamidation (+0.984) on asparagine and glutamine can be set as a variable modification. In non-limiting embodiments, data can be searched against a predetermined database (e.g., a UniProt human database) with peptide-spectrum match (PSMs) and protein-level at 1% false discovery rate (FDR). The FDR can be a multiple hypothesis correction that quantifies the rate of false discoveries or false positive predictions. The signal-to-noise (S/N) measurements of each protein can be normalized so that the sum of the signal for all proteins in each channel can be equivalent to account for equal protein loading. In certain embodiments, the level of the PGD marker and/or additional maker can be measured using enzyme-linked immunosorbent assay (ELISA) assays. For example, ELISA assay can be used to assess PGD maker/additional PGD marker (e.g., KLKB1 protein) concentrations. The ELISA and mass spectrometry-derived protein expression can be compared through the minimum-maximum normalized patient cohort data. The obtained results can be further analyzed for protein expression analysis.

In certain embodiments, the method can include performing protein expression analysis. For example, the difference in protein expression distributions between the prospective and retrospective cohorts can be evaluated (e.g., with the Kolmogorov-Smirnov 2-sample test). The protein expression distribution deviation from the normality test can be from D'Agostino's and Pearson's test, where the normality of a distribution can be rejected at an alpha level p-value. In some embodiments, a differential protein expression signature between PGD and non-PGD patient samples can be calculated. To estimate the association of individual protein levels to PGD, L1-regularized logistic regression models can be calculated for each protein with the sites-of-origin as covariates. For example, about 200 bootstraps (samples with replacement) of the models can be performed to determine a confidence interval for the protein expression association to PGD. The average of the bootstrap distribution for each protein can be used as the differential rank statistic.

In certain embodiments, pathway analysis can be conducted using gene set enrichment analysis (GSEA). For GSEA, the Normalized Enrichment Score (NES) can provide a gene set enrichment compared to all permutations of the gene set enrichment for the protein expression data. The NES can be interpreted as the gene set enrichment score corrected for the size of the gene set and spurious, uninteresting correlations between the gene sets and the expression dataset. The p-value can estimate the probability of seeing an enrichment score as high or higher among the permutation distribution, and the false discovery rate (FDR) can estimate the probability that an enrichment score with a given NES is a false positive finding.

In certain embodiments, the protein prediction contribution can be assessed within each of the pathways and functions from the GSEA analysis. The set of proteins within each pathway and function can be used as features in an L1-regularized logistic regression model (e.g., using a Monte Carlo cross-validation (MCCV) model). For example, if a given pathway A includes a set of 5 proteins, then those 5 proteins can be included as features in the L1-regularized logistic regression model, given the sites-of-origin as covariates.

In certain embodiments, the method can include providing a PGD risk value that can be quantified based on the level of the PGD marker using an adaptive MCCV model. The PGD marker, additional PGD markers, characteristics of the subject, or combinations thereof can be used for calculating the PGD risk value. For example, a Logistic Regression model with L1 regularization for each marker to determine their predictive performance and association to PGD. To estimate the prediction variance and PGD risk value, the MCCV can be used. For example, the PGD prediction probabilities can be compared to the true PGD status to compute the area under the receiver operating characteristic curve (AUROC) and other metrics. From the disclosed model, a possible PGD risk value of 2 can be the log odds risk of PGD for every unit increase of the characteristic. In non-limiting embodiments, bootstrapping analysis (samples with replacement) can be used for analyzing a population distribution for prediction performances, and a permutation analysis can be performed, with random labeling of PGD status in patients, to generate and test prediction metrics from random PGD assignment. In some embodiments, the differences in the bootstrap and permutation distributions, as well as between the 2 bootstrap distributions, with the 2-sample Kolmogorov-Smirnov test can be evaluated.

In certain embodiments, the adaptive MCCV technique can perform prediction of non-PGD as well as PGD. Machine learning models can be used to produce higher probabilities for non-PGD patients, which can result in AUROC values (e.g., less than about 0.5), which can be regarded as a random prediction. The disclosed MCCV technique can sample these patient probabilities to derive an AUROC performance metric and confidence interval. The calculated marker performances can be representative of the model's confidence in predicting the occurrence of PGD. The disclosed machine learning model can be used for predicting the risk of PGD at every iteration of the MCCV technique. In MCCV, patients can be randomly assigned to training and validation sets. Within the training set, the lambda hyperparameter from the machine learning model can be estimated (e.g., using 10-fold cross validation or an appropriate hyperparameter set from the chosen machine learning model). Within each fold, a training set of patients can set the machine learning model parameters and the performance can be assessed on a separate training set. The best performing fold on the testing set can be then chosen to evaluate the machine learning model parameters. The validation set, which has remained unused in the procedure, can be now used to evaluate the performance of the top performing machine learning model (e.g., from the 10-fold cross validation).

In certain embodiments, the method can include providing the disclosed MCCV technique with a training set for machine learning. The disclosed MCCV technique can use a training set to optimize machine learning model hyperparameters to make final predictions of PGD risk. Thus, the size, diversity, and composition of the training set can determine the hyperparameters chosen for the final machine learning model. By utilizing a robust and diverse training set, machine learning model hyperparameters can be chosen for a more accurate and generalizable risk prediction. In non-limiting embodiments, the MCCV technique can be a continuously evolving technique based on the training set. For example, Machine learning and statistical techniques can be used to mitigate confounding in biological enrichment analyses and improve predictive accuracy with modest population size.

In certain embodiments, putative PGD classifiers can be generated from the disclosed MCCV technique and used for the prediction of PGD. The average of the bootstrap distribution of marker importance (beta coefficients) of the disclosed models can be applied to provide PGD risk on new data. Unlike certain classifiers that resemble a simple equation with feature risk coefficients multiplied by the normalized value or indicator of that feature for a patient summed together for a final risk score, the risk score of the putative PGD classifier can undergoe an additional mathematical transformation, a logistic equation, before becoming usable as a clinical risk score. For example, marker A and marker B can have average importance of −1 and −2, respectively. By applying the dot product between the average marker importance of −1 and −2 and a patient's values for markers A and B and applying a logit transformation, the equation results in a probability of PGD risk for each patient. These equations are produced for every two-marker panel. An example equation can be (−0.9946*[pre-transplant Inotrope therapy indicator])+(−2.140*[pre-transplant KLKB1 normalized protein expression value]).

In certain embodiments, the method can include identifying the risk of PGD based on the PGD risk value. Alternation of the level of PGD marker expression can be a predictor of PGD. For example, reduction in KLKB1 can be a predictor of PGD both by itself and in combination with other markers. In non-limiting embodiments, an increase of the makers involved in either inflammation or innate immunity (e.g., PRDX2, MPO, PGLYRP2, and DEFA1) can be a predictor of PGD. In some embodiments, the characteristic of the subject can be evaluated for identifying the PGD risk. For example, the lack of inotrope therapy can be predictive of PGD. Patient's blood type and/or whether the patient has diabetes can also be a risk factor for PGD.

In certain embodiments, the disclosed information related to proteomics and clinical variables can be evaluated through the disclosed model tin increase classification power. For example, KLKB1 combination with inotrope therapy can result in a significant increase in classification power when compared to a combination of KLKB1 and other top-performing proteins. Furthermore, this panel can outperform other composite scores and clinical variables such as the RADIAL score.

In certain embodiments, the disclosed method can further include assessing an effect of a therapy on the heart transplant by estimating the PGD risk value of the subject before/after the therapy administered to the subject. The therapy can be any use of mechanical support and/or drug therapy (e.g., beta blockers, antiarrhythmics, etc.). In non-limiting embodiments, the heart transplant surgery can be canceled based on the identified PGD risk value. In some embodiments, additional therapy can be administered to the subject to reduce the PGD risk value before or after the heart transplant. For example, KLKB1 activators/blockers, anti-inflammatory agents, or combinations can be administered to the subject to reduce PGD risk value.

In certain embodiments, the disclosed subject matter provides a system for predicting PGD and/or treating/preventing PGD based on the prediction. The system can include one or more processors and one or more computer-readable non-transitory storage media coupled to one or more of the processors. The one or more computer-readable non-transitory storage media can include instructions operable when executed by one or more of the processors to cause the system to collect a sample of the subject, measure a level of a PGD marker from the sample, provide a PGD risk value that can be quantified based on the level of the PGD marker using an adaptive Monte Carlo cross-validation (MCCV) model, and identify the risk of PGD based on the PGD risk value. In non-limiting embodiments, the PGD marker can include plasma kallikrein (KLKB1). In some embodiments, the processor can be an electronic circuitry (e.g., central processing unit, graphics processing unit, digital signal processor, etc.) within a computer/server that can include a non-transitory storage media. In non-limiting embodiments, instructions can include a set of machine languages that a processor can understand and execute.

In certain embodiments, the disclosed processor can be configured to collect or receive the sample of the subject. The sample can include any body fluids of the subject. For example, the sample can include blood, serum, tears, effluent fluids, plasma, urine, semen, saliva, bronchial fluid, cerebral spinal fluid (CSF), amniotic fluid, synovial fluid, lymph, bile, gastric acid, or combinations thereof.

In certain embodiments, the disclosed processor can be configured to receive information related to one or more characteristics of a subject. The characteristic can include the disclosed demographics, biometrics, lab values, medications, hemodynamics, cardiomyopathy, transplant factors, clinical variables or combinations thereof.

In certain embodiments, the disclosed processor can be configured to measure or receive information related to a level of a PGD marker from the sample. In non-limiting embodiments, the PGD marker can include proteins peroxiredoxin 2 (PRDX2), tropomyosin alpha-4 (TPM4), myeloperoxidase (MPO), PGLYRP2, DEFA1, DEFA1B, LDHB, F2, FCGBP, CAT, CFHR5, HIST1H4, GAPDH, LTF, ADIPOQ, HSPA5, plasma kallikrein (KLKB1), or combinations thereof. In non-limiting embodiments, the PGD marker can be KLKB1. In some embodiments, the system can be configured to measure or receive information related to the level of the additional marker from the sample. The additional marker can include PRDX2, TPM4, MPO, PGLYRP2, DEFA1, DEFA1B, LDHB, F2, FCGBP, CAT, CFHR5, HIST1H4, GAPDH, LTF, ADIPOQ, HSPA5, KLKB1, IGHD, IGLV2-11, or combinations thereof.

In certain embodiments, the disclosed processor can be configured to provide the disclosed PGD risk value that can be quantified based on the level of the PGD marker using the disclosed adaptive Monte Carlo cross-validation (MCCV) model. The adaptive MCCV model can assess the level of PGD marker, additional marker, characteristics of the subject, or combinations thereof to provide the PGD risk value. For example, the KLKB1 combination and history of inotrope therapy can be assessed for predicting the PGD risk value.

In non-limiting embodiments, the MCCV model can be a continuously evolving model. For example, the processor can include a machine learning program, which can mitigate confounding in biological enrichment analyses and improve predictive accuracy with modest population size. The MCCV model can be improved by providing a training set for machine learning. Training sets can include matched patients (e.g., one patient group that had PGD and one group that did not have PGD but both patients groups were similar age and the same sex). Other criterion can be a number of patients in the training set. In non-limiting embodiments, the processor can be configured to identify the risk of PGD based on the calculated PGD risk value.

In certain embodiments, the processor can be configured to assess an effect of a therapy on the heart transplant by estimating the PGD risk value of the subject. In non-liming embodiments, the processor can provide further recommendations or instructions for additional treatment for the subject based on the PGD risk value. For example, the processor can recommend canceling the heart transplant based on the identified PGD risk value. The processor can recommend additional therapy (e.g., KLKB1 activators, anti-inflammatory agents, or combinations) for reducing the PGD risk value before or after the heart transplant.

In certain embodiments, the disclosed subject matter provides methods for predicting post-transplant survival of a subject seeking an organ transplant. An exemplary method can include, collecting a sample from the subject, measuring a level of a marker predictive of post-transplant survival in the sample, providing a transplant risk value and predicting the likelihood of post-transplant survival based on the transplant risk value.

In certain embodiments, predicting post-transplant survival can identify a risk of primary graft dysfunction (PGD).

In certain embodiments, as shown in FIG. 1, the sample can be collected from a subject. In non-limiting embodiments, the sample can include any body fluids of the subject. For example, the sample can include blood, serum, tears, effluent fluids, plasma, urine, semen, saliva, bronchial fluid, cerebral spinal fluid (CSF), amniotic fluid, synovial fluid, lymph, bile, gastric acid, or combinations thereof.

In certain embodiments, the method can include obtaining one or more characteristics of the subject. The characteristic can include demographics, biometrics, lab values, medications, hemodynamics, cardiomyopathy, transplant factors, clinical variables or combinations thereof. For example, the demographics can include body mass index (BMI), blood type, age, sex, history of tobacco, diabetes, ischemic, or combinations thereof. The cardiomyopathy can include non-ischemic, Adriamycin, amyloid, Chagas, Congenital, Hypertrophic cardiomyopathy, Idiopathic, Myocarditis, Valvular Heart Disease, Viral, Ischemic Time, or combination thereof. The transplant factors can include ventricular assist device, pulmonary artery (PA) diastolic, or a combination thereof. The hemodynamics can include pulmonary artery systolic, PA mean, central venous pressure (CVP), pulmonary capillary wedge pressure (PCWP), creatinine, or a combination thereof The lab values can include an international normalized ratio (INR), total bilirubin, sodium, antiarrhythmic, or combinations thereof. The medications can include beta-blocker, inotrope, CVP/PCWP, or combinations thereof. The clinical variables can include a medical history of the subject (e.g., pre-transplant inotrope therapy). In non-limiting embodiments, the characteristic can be used for calculating RADIAL score and model for end-stage liver disease score (MELD) scores. For example, the MELD score can be derived for each patient using the formula:

3.78×ln[serum bilirubin (mg/dL)]+9.57×ln[serum creatinine (mg/dL)]+6.43 (2)

In non-limiting embodiments, clinical risk scores can include a plurality of risk factors for primary graft dysfunction (e.g., Right atrial pressure>=10 mm Hg, recipient Age>=60 years, Diabetes mellitus, Inotrope dependence, donor Age>=30 years, Length of ischemic time>=240 minutes—i.e., RADIAL score).

In certain embodiments, the level of a post-transplant survival marker can be measured from the sample of the subject. In non-limiting embodiments, the post-transplant survival marker can include proteins prothrombin (F2), anti-plasmin (SERPINF2), Factor IX (F9), carboxypeptidase 2 (CPB2), HGF activator (HGFAC) and low molecular weight kininogen (LK), or combinations thereof. For example, the post-transplant survival marker can be SERPINF2, F9, or LK, or combinations thereof. In some non-limiting embodiments, the post-transplant survival marker can be LK.

In certain embodiments, the level of the post-transplant survival marker and/or additional maker can be measured through various assays. In non-limiting embodiments, the level of the post-transplant survival marker and/or additional maker can be measured using mass spectrometry analysis. For example, microvesicles can be isolated from a sample (e.g., 100 ul) from a subject and homogenized using an MS-compatible lysis buffer. Lysate (e.g., 20 μg) from each sample can be proteolytically cleaved with trypsin and chemically labeled with mass spectrometer detectable quantification reagent.

A reference sample can be generated by pooling equal amounts of microvesicles from each subject to create a protein library for quantification. Samples can be bulk mixed (e.g., at 1:1) across all channels, and bulk mixed samples can be fractionated, and each fraction can be dried. Dried peptides can be dissolved in a solution of 2% acetonitrile/2% formic acid and injected (e.g., in Oribitrap Fusion coupled with the UltiMate™ 3000 RSLCnano system). Fractionated peptides can be separated with an about 5-30% acetonitrile gradient in about 0.1% formic acid over about 70 min.

In non-limiting embodiments, the full MS spectra can be acquired at a resolution of about 120,000. In some embodiments, the method can include selecting the most intense ions (e.g., MS1 ions) for MS2 analysis. MS1 can be the initial ionized sample. These ions can split into smaller fragments usually through collision to generate smaller ions (MS2) and so on (MS3). Each MS represents a greater fragmentation such that the their separation by mass/charge ratio allows to identify individual ions. The isolation width can be set at about 0.7 Da, and isolated precursors can be fragmented by Collision Induced Dissociation (CID) at normalized collision energy (NCE) of 35% and analyzed in the ion trap using “turbo” scan speed. Following the acquisition of each MS2 spectrum, a synchronous precursor selection (SPS) MS3 scan can be collected on the selected ions (e.g., the top 10 most intense ions in the MS2 spectrum). SPS-MS3 precursors can be fragmented by higher energy collision-induced dissociation (HCD) at an normalized collision energy (NCE) of 60% and analyzed.

Raw mass spectrometric data can be analyzed using to perform database search and tandem mass tags (TMT) reporter ions quantification. TMT can be isobaric mass tags that can allow for quantitation of each protein identified in mass spec. TMT tags on lysine residues and peptide N termini (e.g., +229.163 Da) and the carbamidomethylating of cysteine residues (e.g., +57.021 Da) can be set as static modifications, while the oxidation of methionine residues (e.g., +15.995 Da), deamidation (+0.984) on asparagine and glutamine can be set as a variable modification.

In non-limiting embodiments, data can be searched against a predetermined database (e.g., a UniProt human database) with peptide-spectrum match (PSMs) and protein-level at 1% false discovery rate (FDR). The FDR can be a multiple hypothesis correction that quantifies the rate of false discoveries or false positive predictions. The signal-to-noise (S/N) measurements of each protein can be normalized so that the sum of the signal for all proteins in each channel can be equivalent to account for equal protein loading. In certain embodiments, the level of the post-transplant survival maker can be measured using enzyme-linked immunosorbent assay (ELISA) assays. For example, ELISA assay can be used to assess the post-transplant survival marker (e.g., LK protein) concentrations. The ELISA and mass spectrometry-derived protein expression can be compared through the minimum-maximum normalized patient cohort data. The obtained results can be further analyzed for protein expression analysis.

In certain embodiments, the method can include performing protein expression analysis. For example, the difference in protein expression distributions between the prospective and retrospective cohorts can be evaluated (e.g., with the Kolmogorov-Smirnov 2-sample test). The protein expression distribution deviation from the normality test can be from D'Agostino's and Pearson's test, where the normality of a distribution can be rejected at an alpha level p-value.

In some embodiments, a differential protein expression signature between samples collected from surviving and non-surviving patients can be calculated. To estimate the association of individual protein levels to predicting post-transplant survival, L1-regularized logistic regression models can be calculated for each protein with the sites-of-origin as covariates. For example, about 200 bootstraps (samples with replacement) of the models can be performed to determine a confidence interval for the protein expression association to post-transplant survival. The average of the bootstrap distribution for each protein can be used as the differential rank statistic.

In certain embodiments, pathway analysis can be conducted using gene set enrichment analysis (GSEA). For GSEA, the Normalized Enrichment Score (NES) can provide a gene set enrichment compared to all permutations of the gene set enrichment for the protein expression data. The NES can be interpreted as the gene set enrichment score corrected for the size of the gene set and spurious, uninteresting correlations between the gene sets and the expression dataset. The p-value can estimate the probability of seeing an enrichment score as high or higher among the permutation distribution, and the false discovery rate (FDR) can estimate the probability that an enrichment score with a given NES is a false positive finding.

In certain embodiments, the protein prediction contribution can be assessed within each of the pathways and functions from the GSEA analysis. The set of proteins within each pathway and function can be used as features in an L1-regularized logistic regression model (e.g., using a Monte Carlo cross-validation (MCCV) model). For example, if a given pathway A includes a set of 5 proteins, then those 5 proteins can be included as features in the L1-regularized logistic regression model, given the sites-of-origin as covariates.

In certain embodiments, the method can include providing a transplant risk value that can be quantified based on the level of the post-transplant survival marker using an adaptive MCCV model. The post-transplant survival marker, characteristics of the subject, or combinations thereof can be used for calculating the transplant risk value. For example, a Logistic Regression model with L1 regularization for each marker to determine their predictive performance and association to post-transplant survival. To estimate the prediction variance and transplant risk value, the MCCV can be used.

For example, the post-transplant survival prediction probabilities can be compared to the true post-transplant survival status to compute the area under the receiver operating characteristic curve (AUROC) and other metrics. From the disclosed model, a possible transplant risk value of 2 can be the log odds risk for every unit increase of the characteristic. In non-limiting embodiments, bootstrapping analysis (samples with replacement) can be used for analyzing a population distribution for prediction performances, and a permutation analysis can be performed, with random labeling of post-transplant survival status in patients, to generate and test prediction metrics from random transplant risk assignment. In some embodiments, the differences in the bootstrap and permutation distributions, as well as between the 2 bootstrap distributions, with the 2-sample Kolmogorov-Smirnov test can be evaluated.

In certain embodiments, the adaptive MCCV technique can perform prediction of risk to survival following a transplant. Machine learning models can be used to produce higher probabilities for non-risk patients, which can result in AUROC values (e.g., less than about 0.5), which can be regarded as a random prediction. The disclosed MCCV technique can sample these patient probabilities to derive an AUROC performance metric and confidence interval. The calculated marker performances can be representative of the model's confidence in predicting the risk to survival. The disclosed machine learning model can be used for predicting the risk to survival at every iteration of the MCCV technique.

In MCCV, patients can be randomly assigned to training and validation sets. Within the training set, the lambda hyperparameter from the machine learning model can be estimated (e.g., using 10-fold cross validation or an appropriate hyperparameter set from the chosen machine learning model). Within each fold, a training set of patients can set the machine learning model parameters and the performance can be assessed on a separate training set. The best performing fold on the testing set can be then chosen to evaluate the machine learning model parameters. The validation set, which has remained unused in the procedure, can be now used to evaluate the performance of the top performing machine learning model (e.g., from the 10-fold cross validation).

In certain embodiments, the method can include providing the disclosed MCCV technique with a training set for machine learning. The disclosed MCCV technique can use a training set to optimize machine learning model hyperparameters to make final predictions of transplant risk. Thus, the size, diversity, and composition of the training set can determine the hyperparameters chosen for the final machine learning model. By utilizing a robust and diverse training set, machine learning model hyperparameters can be chosen for a more accurate and generalizable risk prediction. In non-limiting embodiments, the MCCV technique can be a continuously evolving technique based on the training set. For example, Machine learning and statistical techniques can be used to mitigate confounding in biological enrichment analyses and improve predictive accuracy with modest population size.

In certain embodiments, putative transplant risk classifiers can be generated from the disclosed MCCV technique and used for the prediction of transplant risk. The average of the bootstrap distribution of marker importance (beta coefficients) of the disclosed models can be applied to provide PGD risk on new data. Unlike certain classifiers that resemble a simple equation with feature risk coefficients multiplied by the normalized value or indicator of that feature for a patient summed together for a final risk score, the risk score of the putative transplant risk classifier can undergoe an additional mathematical transformation, a logistic equation, before becoming usable as a clinical risk score.

For example, marker A and marker B can have average importance of −1 and −2, respectively. By applying the dot product between the average marker importance of −1 and −2 and a patient's values for markers A and B and applying a logit transformation, the equation results in a probability of transplant risk for each patient. These equations are produced for every two-marker panel. An example equation can be (−0.9946*[pre-transplant Inotrope therapy indicator])+(−2.140*[pre-transplant KLKB1 normalized protein expression value]).

In certain embodiments, the method can include identifying the likelihood of post-transplant survival based on the transplant risk value. Alternation of the level of likelihood of post-transplant survival marker expression can be a predictor of transplant risk. In some embodiments, a level of LK outside a distribution of values for LK established in a survival cohort can be a predictor of post-transplant survival both by itself and in combination with other markers. For example, a lower value of LK compared to the distribution of values established in a survival cohort can be a predictor of post-transplant survival. In other embodiments, a level of LK outside a distribution of values for LK established in a survival cohort and, a level of F2, SERPINF2, F9, CPB2, or HGFAC outside a distribution of values for F2, SERPINF2, F9, CPB2, or HGFAC respectively established in a survival cohort, can be a predictor of post-transplant survival. For example, a lower value of LK compared to the distribution of values established in a survival cohort and, a higher value of F2, SERPINF2, F9, CPB2, or HGFAC compared to the distribution of values established in a survival cohort can be a predictor of post-transplant survival.

In some embodiments, the characteristic of the subject can be evaluated for identifying the transplant risk. For example, the lack of inotrope therapy can be predictive of transplant risk. Patient's blood type and/or whether the patient has diabetes can also be a risk factor for post-transplant survival.

In certain embodiments, the disclosed information related to proteomics and clinical variables can be evaluated through the disclosed model tin increase classification power. For example, LK combination with inotrope therapy can result in a significant increase in classification power when compared to a combination of LK and other top-performing proteins. Furthermore, this panel can outperform other composite scores and clinical variables such as the RADIAL score.

In certain embodiments, the disclosed method can further include assessing an effect of a therapy on the heart transplant by estimating the transplant risk value of the subject before/after the therapy administered to the subject. The therapy can be any use of mechanical support and/or drug therapy (e.g., beta blockers, antiarrhythmics, etc.). In non-limiting embodiments, the heart transplant surgery can be canceled based on the identified transplant risk value. In some embodiments, additional therapy can be administered to the subject to reduce the transplant risk value before or after the heart transplant. For example, LK activators/blockers, anti-inflammatory agents, or combinations thereof can be administered to the subject to reduce transplant risk value.

In certain embodiments, the disclosed subject matter provides a system for predicting post-transplant survival of a subject seeking an organ transplant and/or treating/preventing transplant risk based on the prediction. The system can include one or more processors and one or more computer-readable non-transitory storage media coupled to one or more of the processors. The one or more computer-readable non-transitory storage media can include instructions operable when executed by one or more of the processors to cause the system to collect a sample from the subject, measure a level of a marker predictive of post-transplant survival from the sample, provide a transplant risk value that can be quantified based on the level of the post-transplant survival marker using an adaptive Monte Carlo cross-validation (MCCV) model, and identify the tranaplantation risk based on the level of the post-transplant survival marker.

In some embodiments, the processor can be an electronic circuitry (e.g., central processing unit, graphics processing unit, digital signal processor, etc.) within a computer/server that can include a non-transitory storage media. In non-limiting embodiments, instructions can include a set of machine languages that a processor can understand and execute.

In certain embodiments, the disclosed processor can be configured to collect or receive the sample of the subject. The sample can include any body fluids of the subject. For example, the sample can include blood, serum, tears, effluent fluids, plasma, urine, semen, saliva, bronchial fluid, cerebral spinal fluid (CSF), amniotic fluid, synovial fluid, lymph, bile, gastric acid, or combinations thereof.

In certain embodiments, the disclosed processor can be configured to receive information related to one or more characteristics of a subject. The characteristic can include the disclosed demographics, biometrics, lab values, medications, hemodynamics, cardiomyopathy, transplant factors, clinical variables or combinations thereof.

In certain embodiments, the disclosed processor can be configured to measure or receive information related to a level of the post-transplant survival marker marker from the sample. In non-limiting embodiments, the post-transplant survival marker can include prothrombin (F2), anti-plasmin (SERPINF2), Factor IX (F9), carboxypeptidase 2 (CPB2), HGF activator (HGFAC), low molecular weight kininogen (LK) or combinations of these. In some non-limiting embodiments, the post-transplant survival marker can be SERPINF2, F9, or LK, or combinations thereof. In some non-limiting embodiments, the post-transplant survival marker can be LK.

In certain embodiments, the disclosed processor can be configured to provide the disclosed transplant risk value that can be quantified based on the level of the post-transplant survival marker using the disclosed adaptive Monte Carlo cross-validation (MCCV) model. The adaptive MCCV model can assess the level of post-transplant survival marker, characteristics of the subject, or combinations thereof to provide the transplant risk value. For example, a combination of LK levels and history of inotrope therapy can be assessed for predicting the transplant risk value.

In non-limiting embodiments, the MCCV model can be a continuously evolving model. For example, the processor can include a machine learning program, which can mitigate confounding in biological enrichment analyses and improve predictive accuracy with modest population size. The MCCV model can be improved by providing a training set for machine learning. Training sets can include matched patients (e.g., one patient group that had a risk to post-transplant survival and one group that did not have a risk to post-transplant survival but both patients groups were similar age and the same sex). Other criterion can be a number of patients in the training set. In non-limiting embodiments, the processor can be configured to identify the risk to post-transplant survival based on the calculated transplant risk value.

In certain embodiments, the processor can be configured to assess an effect of a therapy on the heart transplant by estimating the transplant risk value for the subject. In non-liming embodiments, the processor can provide further recommendations or instructions for additional treatment for the subject based on the transplant risk value. For example, the processor can recommend canceling the heart transplant based on the identified transplant risk value. The processor can recommend additional therapy (e.g., LK activators, anti-inflammatory agents, or combinations) for reducing the transplant risk value before or after the heart transplant.

### EXAMPLES

### Example 1: Plasma Kallikrein Predicts Primary Graft Dysfunction after Heart Transplant

Primary graft dysfunction (PGD) after heart transplant can be defined as idiopathic ventricular dysfunction during the immediate post-transplant period. PGD can affect either or both ventricles simultaneously and be graded from mild to severe depending on the amount of compensatory support required. The International Society for Heart and Lung Transplantation reported that PGD is the leading cause of death within 30 days after transplant. Identifying predictive factors of PGD has the potential to improve risk stratification, organ allocation, and post-operative care, as well as increase the understanding of the etiology of PGD. However, a risk model based solely on pre-transplant recipient factors remains elusive.

Molecular biomarkers can be predictive and robust for many diseases. A rich and underexplored source of potential prognostic biomarkers can be contained in extracellular vesicles. In addition to diagnostic potential, extracellular vesicles can be stable, easily extracted from patient blood, and be used in the prediction of heart disease. The disclosed subject matter provides techniques for a multi-institutional cohort analysis to predict PGD using machine learning to identify combinations of serum microvesicle proteomics and clinical characteristics.

Patient cohorts: patient blood samples were prospectively recruited between 2014 and 2016. Patient blood samples were retrospectively collected from biobanks at Cedars-Sinai hospital (Cedars) and Pitié-Salpêtrière University Hospital (Paris). Only severe PGD by ISHLT definition was included. Patients undergoing re-transplant were excluded. The initial cohort for PGD prediction was comprised of PGD samples matched to non-PGD samples by age and gender. In order to calculate more clinically relevant predictive values, the validation ELISA cohort included consecutive patients undergoing a transplant. Human subjects protocol was approved by each institution's IRB, and patients provided informed consent. Patient characteristics were collected, including demographics, biometrics, labs, medications and hemodynamics. PGD status was defined per ISHLT guidelines.

Mass spectrometry analysis: patient samples from each site were collected for processing. Each patient cohort was processed independently. The total microvesicle was isolated from serum. Each sample was proteolytically cleaved with trypsin and chemically labeled with TMT10plex isobaric mass tags separately. MS spectra were acquired with an Orbitrap Fusion Tribrid Mass Spectrometer (Thermo Scientific), and raw spectrometric data were analyzed using Proteome Discoverer.

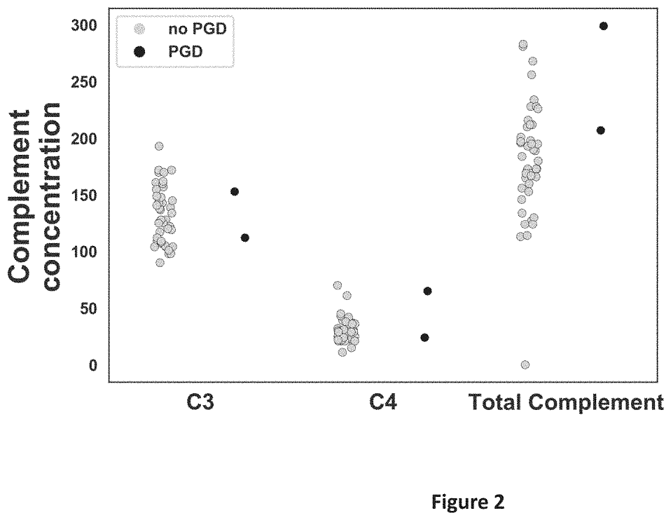

Protein expression analysis: a differential protein expression signature between PGD and non-PGD patient samples was calculated (FIG. 2). The protein association calculated was used as the differential rank statistic for pathway analysis using gene set enrichment analysis (GSEA). FIG. 2 shows the clinical diagnostic ELISA tests for C3, C4, total complement proteins. C3 are mg/dl, C4 are mg/dl, and total complement are U/ml.

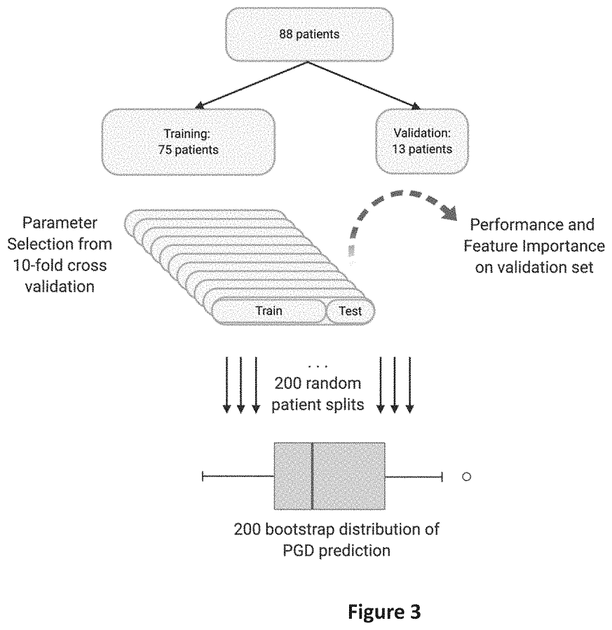

PGD prediction: a Logistic Regression model with L1 regularization was used for each marker to determine their predictive performance and association to PGD (see FIG. 3). FIG. 3 shows a Monte Carlo Cross-Validation (MCCV) Prediction diagram. PGD prediction strategy for estimating the prediction of clinical and protein markers toward the occurrence of PGD post-heart transplant are shown in FIG. 3. An L1-regularized logistic regression model predicted post-transplant PGD using each pre-transplant clinical and protein marker's value distribution. The prediction scheme estimates the variance of prediction using different patient splits of the patient population. Patients were randomly assigned to training (75 patients) and validation (13 patients) sets. Within the training set, model parameters were estimated using 10-fold cross-validation. Within each fold, 64 patient data set the model parameters, and 11 patient data test the model performance.

The model parameters with the best prediction performance can be used as initial parameters to train the model on all 75 patients in the training set. The 13 patients in the validation set, which have been set aside throughout the procedure, were now used to evaluate the model's prediction performance. The importance of the marker towards the prediction on the validation patient data is collected from the beta coefficients of the logistic regression model. The end result is a 200 bootstrap confidence interval of PGD prediction performance and importance for each of the clinical and protein markers controlling for the patient's site-of-origin. 200 random patient splits were computed following this prediction paradigm for comparison to a random prediction distribution.

Confidence intervals were generated from predicted patient probabilities by taking 50 bootstraps and calculating the mean and 95% confidence interval. To estimate the prediction variance, Monte Carlo cross-validation (MCCV) was used. The PGD prediction probabilities were compared to the true PGD status to compute the area under the receiver operating characteristic curve (AUROC) and other metrics. Bootstrapping analysis (samples with replacement) resulted in population distribution for prediction performances, and a permutation analysis was similarly performed, with random labeling of PGD status in patients, to generate and test prediction metrics from random PGD assignment. Differences were evaluated in the bootstrap and permutation distributions, as well as between the 2 bootstrap distributions, with the 2-sample Kolmogorov-Smirnov test. Statistics followed by the use of bracket notation indicated reporting of the average statistic and its 95% confidence interval. The average statistic and standard errors were noted when reporting Student t-test results.

KLKB1 ELISA assay heart transplant patients: enzyme-linked immunosorbent assay (ELISA) (Abcam) was used to assess KLKB1 protein concentration in a validation cohort of pre-transplant serum prospectively collected in 65 consecutive patients at CUIMC. To be able to compare ELISA and mass spectrometry derived protein expression, the patient cohort data was minimum-maximum normalized before application of the MCCV strategy for all predictions.

Patient clinical characteristics: in total, 88 patients who underwent heart transplantation between 2014 and 2016 at Cedars Sinai Medical Center (n=43), Pitié-Salpêtrière University Hospital (n=29) and Columbia University Irving Medical Center (n=16) were used for the initial proteomic and clinical characteristic analysis (Table 1).

TABLE 1
Clinical Characteristics.

| | N | PGD = No 46 | | PGD = Yes 42 | | P value |
|---|---|---|---|---|---|---|
| Patient characteristics | Age (mean (SD)) | 54.90 | (13.43) | 58.43 | (10.13) | 0.171 |
| | BMI (mean (SD)) | 25.11 | (4.07) | 26.46 | (5.07) | 0.171 |
| | Blood Type (%) | | | | | |
| | A | 19 | (41.3) | 15 | (35.7) | |
| | AB | 5 | (10.9) | 3 | (7.1) | |
| | B | 8 | (17.4) | 5 | (11.9) | |
| | 0 | 14 | (30.4) | 19 | (45.2) | |
| | Donor Age (mean (SD)) | 39.98 | (13.92) | 40.95 | (13.44) | 0.74 |
| | Sex = F (%) | 14 | (30.4) | 13 | (31.0) | 1 |
| | History of Tobacco Use = Y (%) | 15 | (32.6) | 16 | (38.1) | 0.753 |
| | Autoimmune Diseases = Y (%) | 2 | (4.4) | 1 | (2.2) | |
| | Diabetes = Y (%) | 14 | (30.4) | 15 | (35.7) | 0.765 |
| Cardiomyopathy | Ischemic = Y (%) | 14 | (30.4) | 18 | (42.9) | 0.323 |
| | Non-Ischemic (%) | | | | | 0.271 |
| | Adriamycin | 0 | (0.0) | 1 | (2.4) | |
| | Amyloid | 2 | (4.3) | 0 | (0.0) | |
| | Chagas | 0 | (0.0) | 1 | (2.4) | |
| | Congenital | 0 | (0.0) | 1 | (2.4) | |
| | Hypertrophic cardiomyopathy | 1 | (2.2) | 0 | (0.0) | |
| | Idiopathic | 28 | (60.9) | 19 | (45.2) | |
| | Myocarditis | 0 | (0.0) | 1 | (2.4) | |
| | Valvular Heart Disease | 0 | (0.0) | 1 | (2.4) | |
| | Viral | 1 | (2.2) | 0 | (0.0) | |
| Transplant factors | Ischemic Time (minutes (SD)) | 156.50 | (62.97) | 169.09 | (53.02) | 0.315 |
| | Ventricular Assist Device = Y (%) | 8 | (17.4) | 13 | (31.0) | 0.215 |
| Hemodynamics | PA Diastolic (mean (SD)) mmHg | 20.73 | (6.76) | 20.38 | (7.79) | 0.823 |
| | PA Systolic (mean (SD)) mmHg | 42.85 | (12.46) | 45.48 | (15.07) | 0.373 |
| | PA Mean (mean (SD)) mmHg | 29.31 | (8.31) | 31.28 | (9.04) | 0.289 |
| | CVP (mean (SD)) mmHg | 9.49 | (5.28) | 9.97 | (5.18) | 0.671 |
| | PCWP (mean (SD)) mmHg | 19.81 | (7.78) | 20.08 | (8.88) | 0.877 |
| Lab values | Creatinine (mean (SD)) mg/dL | 1.36 | (1.14) | 1.25 | (0.43) | 0.558 |
| | INR (mean (SD)) | 1.48 | (0.49) | 1.65 | (0.74) | 0.196 |
| | Tbili (mean (SD)) mg/dL | 0.93 | (0.48) | 0.79 | (0.50) | 0.205 |
| | Sodium (mean (SD)) mEq/L | 137.50 | (4.64) | 136.90 | (5.08) | 0.565 |
| Medications | Antiarrhythmic Use = Y (%) | 22 | (47.8) | 25 | (59.5) | 0.376 |
| | Beta Blocker = Y (%) | 24 | (52.2) | 30 | (71.4) | 0.102 |
| | Inotrope = Y (%) | 30 | (65.2) | 14 | (33.3) | 0.006 |
| Composite Scores | CVP/PCWP (mean (SD)) | 0.49 | (0.25) | 0.55 | (0.28) | 0.273 |
| | MELD (mean (SD)) | 13.57 | (4.74) | 14.31 | (5.18) | 0.483 |
| | Radial Score (mean (SD)) | 2.57 | (1.28) | 2.24 | (0.98) | 0.185 |

Recipient characteristics at the time of transplant unless otherwise specified. Significance evaluated with a continuity-corrected chi-squared test for categorical characteristics and t-test for continuous characteristic: primary graft dysfunction (PGD), body mass index (BMI), pulmonary artery (PA), central venous pressure (CVP), pulmonary capillary wedge pressure (PCWP), international normalized ratio (INR), total bilirubin (TBili), and model for end-stage liver disease score (MELD).

There were 37 different pre-transplant clinical characteristics across all the patients, including PGD status (Table 2). Prior inotrope therapy significantly differed (linear model with and without site-of-origin p-values=0.002 and 0.003) between PGD and non-PGD (Table 1).

TABLE 2
Baseline characteristics of patients.

| | Recipient Clinical Characteristics | | | | | | |
|---|---|---|---|---|---|---|---|
| | Number of Patients | Cedar | | Columbia | | Paris | |
| | PGD = Y (%) | 43 | | 16 | | 29 | |
| | Age (mean (SD)) | 21 | (48.8) | 8 | (50.0) | 13 | (44.8) |
| Patient characteristics | BMI (mean (SD)) | 57.95 | (12.76) | 56.50 | (10.28) | 54.60 | (11.91) |
| | Blood Type (%) | 25.49 | (4.96) | 28.10 | (3.58) | 24.86 | (4.22) |
| | A | | | | | | |
| | AB | 17 | (39.5) | 6 | (37.5) | 11 | (37.9) |
| | B | 4 | (9.3) | 3 | (18.8) | 1 | (3.4) |
| | O | 5 | (11.6) | 2 | (12.5) | 6 | (20.7) |
| | Donor Age (mean (SD)) | 17 | (39.5) | 5 | (31.2) | 11 | (37.9) |
| | Sex = F (%) | 36.49 | (12.21) | 38.50 | (12.18) | 47.38 | (14.05) |
| | History of Tobacco Use = Y (%) | 15 | (34.9) | 2 | (12.5) | 10 | (34.5) |
| | Diabetes = Y (%) | 2 | (4.7) | 11 | (68.8) | 18 | (62.1) |
| | Ischemic = Y (%) | 12 | (27.9) | 7 | (43.8) | 10 | (34.5) |
| Cardiomyopathy | Non-Ischemic (%) | 12 | (27.9) | 8 | (50.0) | 12 | (41.4) |
| | Adriamycin | | | | | | |
| | Amyloid | 1 | (2.3) | 0 | (0.0) | 0 | (0.0) |
| | Chagas | 2 | (4.7) | 0 | (0.0) | 0 | (0.0) |
| | Congenital | 0 | (0.0) | 1 | (6.2) | 0 | (0.0) |
| | Hypertrophic cardiomyopathy | 1 | (2.3) | 0 | (0.0) | 0 | (0.0) |
| | Idiopathic | 1 | (2.3) | 0 | (0.0) | 0 | (0.0) |
| | Myocarditis | 23 | (53.5) | 7 | (43.8) | 17 | (58.6) |
| | Valvular Heart Disease | 1 | (2.3) | 0 | (0.0) | 0 | (0.0) |
| | Viral | 1 | (2.3) | 0 | (0.0) | 0 | (0.0) |
| | Ischemic Time (minutes (SD)) | 1 | (2.3) | 0 | (0.0) | 0 | (0.0) |
| Transplant factors | Ventricular Assist Device = Y (%) | 148.33 | (65.49) | 178.36 | (44.66) | 174.79 | (50.02) |
| | PA Diastolic (mean (SD)) mmHg | 8 | (18.6) | 11 | (68.8) | 2 | (6.9) |
| Hemodynamics | PA Systolic (mean (SD)) mmHg | 19.85 | (6.35) | 15.62 | (7.72) | 24.35 | (6.37) |
| | PA Mean (mean (SD)) mmHg | 41.37 | (12.08) | 36.81 | (12.59) | 52.17 | (13.21) |
| | CVP (mean (SD)) mmHg | 29.32 | (6.93) | 24.00 | (9.14) | 35.07 | (8.31) |
| | PCWP (mean (SD)) mmHg | 10.54 | (5.27) | 7.00 | (5.79) | 10.00 | (4.39) |
| | Creatinine (mean (SD)) mg/dL | 18.99 | (7.16) | 15.38 | (10.29) | 23.87 | (7.06) |
| Lab values | INR (mean (SD)) | 1.41 | (1.21) | 1.28 | (0.32) | 1.18 | (0.32) |
| | TBILI (mean (SD)) mg/dL | 1.47 | (0.56) | 1.71 | (0.73) | 1.60 | (0.66) |
| | Sodium (mean (SD)) MEq/L | 0.75 | (0.29) | 0.53 | (0.31) | 1.21 | (0.60) |
| | Antiarrhythmic = Y (%) | 136.46 | (4.16) | 140.31 | (5.16) | 136.62 | (5.07) |
| Medications | Beta Blocker = Y (%) | 27 | (62.8) | 7 | (43.8) | 13 | (44.8) |
| | Inotrope = Y (%) | 25 | (58.1) | 14 | (87.5) | 15 | (51.7) |
| | CVP/PCWP (mean (SD)) | 23 | (53.5) | 5 | (31.2) | 16 | (55.2) |
| Composite Scores | MELD (mean (SD)) | 0.57 | (0.29) | 0.52 | (0.29) | 0.44 | (0.21) |
| | RADIALScore (mean (SD)) | 13.47 | (5.18) | 14.44 | (5.23) | 14.31 | (4.50) |

In a multivariate model including all characteristics, only pre-transplant inotrope therapy associates with PGD (Table 3).

TABLE 3
Cellular enrichment of identified proteins.

| | Gene Ontology Cellular Component | | | |
|---|---|---|---|---|
| | Pathway | Observed | Background | False Discovery |
| ID | Description | Gene Count | Gene Count | Rate |
| GO:0005576 | extracellular region | 158 | 2505 | 1.38E−111 |
| GO:0044421 | extracellular region part | 117 | 1375 | 1.46E−84 |
| GO:0005615 | extracellular space | 110 | 1134 | 6.98E−84 |
| GO:0060205 | cytoplasmic vesicle lumen | 55 | 340 | 4.94E−48 |
| GO:0034774 | secretory granule lumen | 49 | 323 | 2.50E−41 |

Patient blood microvesicle proteomic characteristics: serum microvesicle protein spectra were obtained in at least triplicate for each patient (322 total replicates) (FIG. 1A). The identified proteins were enriched in micro-vesicle and extracellular components (Table 3). Table 4 is a Table sorted by Area Under the Receiver Operating Characteristic curve (AUROC). The beta coefficients of the models were exponentiated to odds shown below. The lower and upper bounds indicate the 95% confidence interval. AUROC average>0.5, Bonferroni corrected p-value<0.001, beta coefficient 95% CI not including the null association, and permutation beta coefficient 95% CI including the null association. Significant clinical characteristics were highlighted.

TABLE 4
Prediction statistics of significant protein markers and clinical characteristics.

| | AUROC | | AUROC | Feature | Odds | | Odds |
|---|---|---|---|---|---|---|---|
| | lower | AUROC | upper | Importance | lower | Odds | upper |
| Marker | bound | average | bound | P-value | bound | average | bound |
| KLKB1 | 0.6293 | 0.6444 | 0.6655 | 8.70E−84 | 0.0592 | 0.1959 | 0.3663 |
| PRDX2 | 0.6075 | 0.6281 | 0.6452 | 1.99E−86 | 3.7618 | 10.7457 | 29.2799 |
| TPM4 | 0.6051 | 0.6229 | 0.6442 | 1.99E−86 | 4.5053 | 10.8144 | 22.427 |
| MPO | 0.5823 | 0.6004 | 0.6195 | 1.16E−84 | 2.7906 | 7.685 | 15.6071 |
| PGLYRP2 | 0.5667 | 0.5895 | 0.6073 | 4.76E−82 | 0.1245 | 0.3011 | 0.6029 |
| DEFA1; | 0.5708 | 0.5884 | 0.6044 | 8.83E−78 | 2.0527 | 5.6507 | 13.6866 |
| DEFA1B | | | | | | | |
| LDHB | 0.5559 | 0.5761 | 0.5915 | 6.11E−77 | 1.5371 | 3.9759 | 9.3158 |
| Inotrope | 0.5387 | 0.5618 | 0.58 | 1.81E−78 | 0.3043 | 0.4342 | 0.6033 |
| therapy | | | | | | | |
| F2 | 0.5395 | 0.5593 | 0.5791 | 6.11E−77 | 0.1577 | 0.3827 | 0.7748 |
| FCGBP | 0.5387 | 0.5587 | 0.5774 | 1.99E−86 | 2.3899 | 4.5954 | 7.9145 |
| CAT | 0.527 | 0.5452 | 0.5667 | 2.50E−80 | 2.4282 | 5.7301 | 13.3989 |
| CFHR5 | 0.5248 | 0.5438 | 0.5681 | 8.33E−73 | 1.216 | 2.8542 | 6.5664 |
| HIST1H4 | 0.5196 | 0.5391 | 0.5573 | 2.50E−80 | 1.9951 | 4.5833 | 8.373 |
| family | | | | | | | |
| GAPDH | 0.5151 | 0.5345 | 0.5574 | 2.23E−70 | 1.2203 | 3.1299 | 9.258 |
| LTF | 0.511 | 0.5304 | 0.5478 | 5.42E−72 | 1.1191 | 3.0995 | 5.8155 |
| ADIPOQ | 0.5011 | 0.5163 | 0.5307 | 3.49E−71 | 0.218 | 0.467 | 0.7907 |
| HSPA5 | 0.4986 | 0.5132 | 0.5286 | 2.44E−63 | 1.0756 | 2.0785 | 4.6261 |

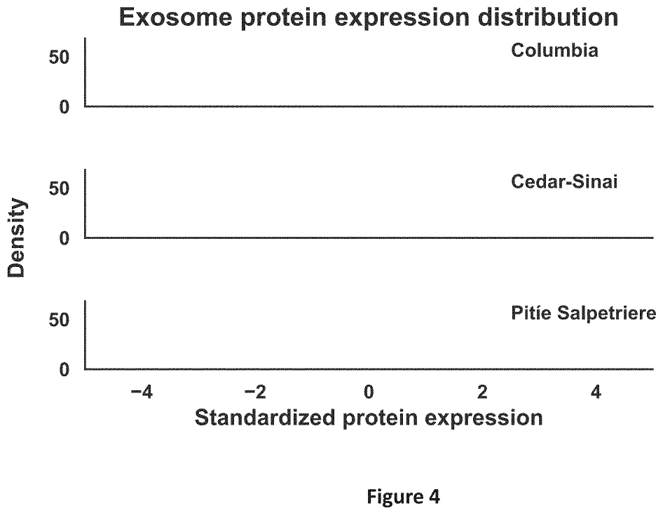

Protein expression in the three patient cohorts (FIG. 4) does not follow a normal distribution (Omnibus test of normality p-values<<0.001). FIG. 4 shows exosome protein expression distributions for the patient cohorts. The individual patient expression distributions in each cohort were superimposed to represent each patient's individual contribution to the whole cohort protein expression distribution. The Columbia cohort was significantly different from Cedars-Sinai (Kolmogorov Smirnov test p-value<3.19E-08) and from Pitié-Salpêtrière (p-value=8.70E-06). Protein expression was statistically different between the 2 retrospective patient cohorts (p-value=0.030).

In total, 681 unique proteins were identified with 345 identified proteins present in every cohort of the patient cohorts and 80 proteins were not identified in at least one patient (FIG. 1B). There were 81 identified immunoglobulin proteins that were not included in the analysis. Additionally, three proteins did not have corresponding gene name annotations. A final set of 181 proteins, which were identified in every patient across all patient cohorts, were used in downstream analyses (FIG. 1C).

Prediction of post-transplant PGD using pre-transplant clinical and protein markers: the prediction of post-transplant PGD in patients was investigated using clinical and protein markers derived prior to transplant. Monte Carlo cross-validation (MCCV; FIG. 3) and permutation analysis was employed to calculate the prediction and significance of each clinical and protein marker in predicting PGD.

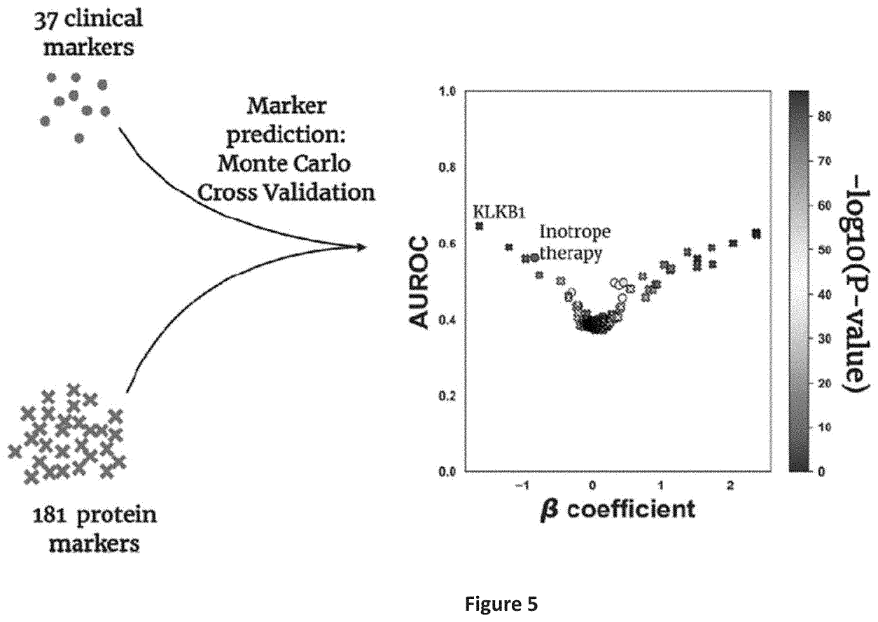

Overall, the expression of all protein markers did not significantly outperform (AUROC 0.4119±0.05473 vs 0.3751±0.04712 independent 2-sample t-test p-value=0.9147) nor were more influential (odds 1.3477±1.3324 vs 1.0544±0.2115 p-value=0.1819) than all clinical characteristics in predicting the post-transplant occurrence of PGD (FIG. 5). Individually, 16 proteins and 1 clinical characteristic were significantly predictive of PGD occurrence (AUROC>0.5, Bonferroni-corrected p-value<0.001, beta coefficient 95% CI not including the null association, and permutation beta coefficient 95% CI including the null association). In Table 5, panels were significantly predictive when the performance upper bound of KLKB1 was lower than the lower bound of the two marker panels. The performance coefficient of variation was calculated by taking the log base 10 of the ratio between the average performance across all and within each cohort and the variation between them.

TABLE 5
Two marker panels significantly outperforming
top individual predictive marker KLKB1.

| | | | | Performance |
|---|---|---|---|---|
| | AUROC | | AUROC | coefficient of |
| | lower | AUROC | upper | variation |
| Panel | bound | average | bound | (log10) |
| KLKB1 and inotrope therapy | 0.7020 | 0.7181 | 0.7372 | 2.569 |
| KLKB1 and TPM4 | 0.6994 | 0.7152 | 0.7341 | 3.008 |
| TPM4 and PGLYRP2 | 0.6933 | 0.7108 | 0.7257 | 1.410 |
| KLKB1 and PRDX2 | 0.6790 | 0.6967 | 0.7107 | 1.789 |
| KLKB1 and DEFA1; DEFA1B | 0.6685 | 0.6870 | 0.7024 | 1.792 |

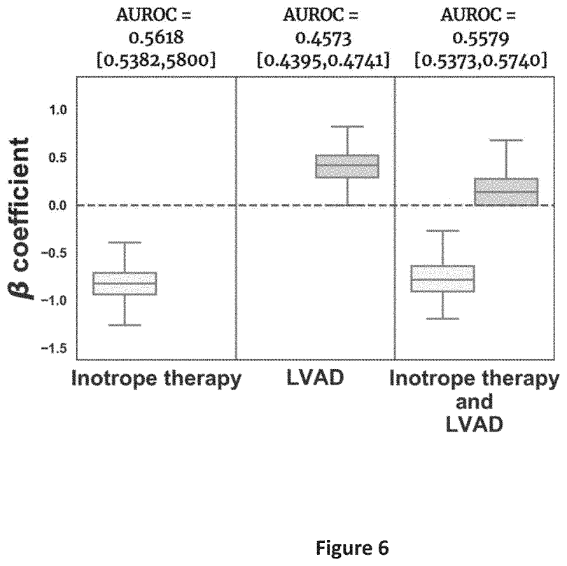

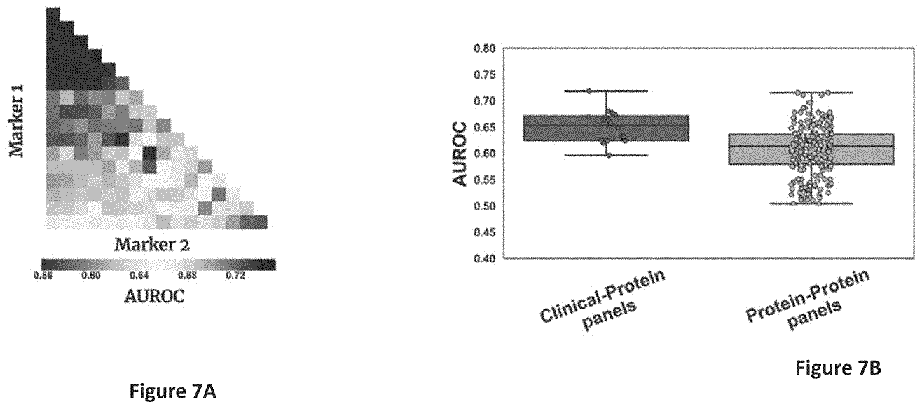

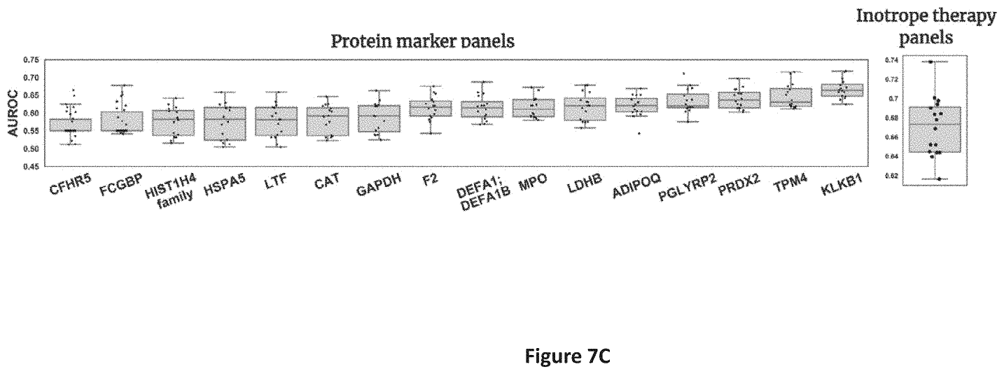

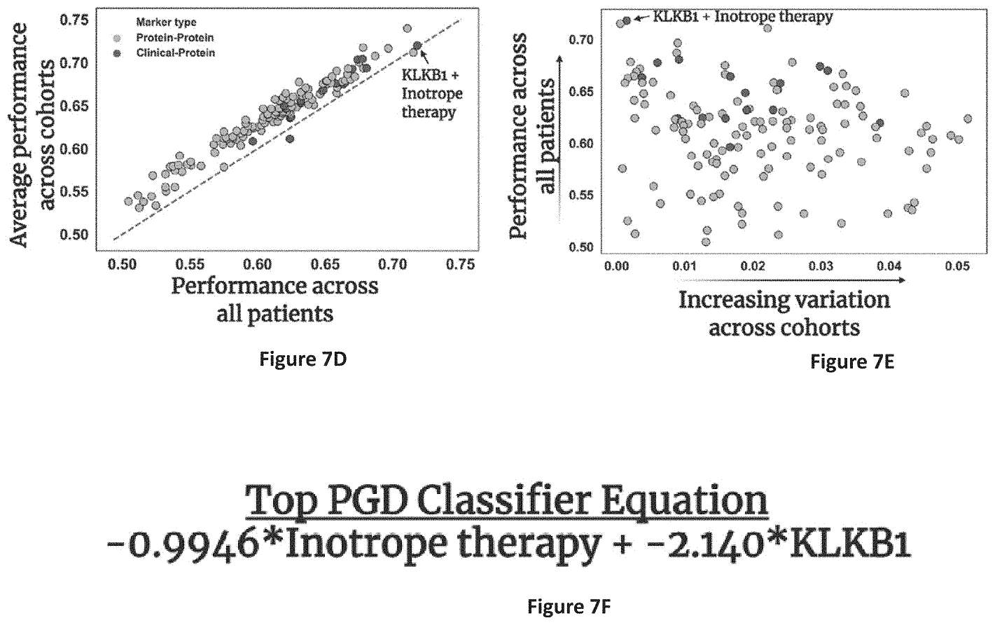

The most predictive protein marker was plasma kallikrein (KLKB1) (AUROC 0.6444 [0.6293, 0.6655]; odds 0.1959 [0.0592, 0.3663]) where decreased expression of KLKB1 was significantly predictive of PGD status. The next most predictive markers (AUROC>0.6) were the proteins peroxiredoxin 2 (PRDX2), tropomyosin alpha-4 (TPM4), and myeloperoxidase (MPO), where increased expression of each was significantly predictive of PGD status (Table 5). With respect to clinical factors, the absence of pre-transplant inotrope therapy was significantly predictive of PGD on its own, albeit modestly. (AUROC 0.5618 [0.5387, 0.5800]; average odds 0.4342 [0.3043, 0.6033]). Notably the presence of mechanical support was not predictive (AUROC 0.4753 [0.4395, 0.4741], odds 1.192 [1.000, 1.781],) nor did it attenuate the predictive performance of pre-transplant inotrope therapy towards PGD (FIG. 6). FIG. 5 shows that pre-transplant inotrope therapy can be predictive of PGD independent of a left ventricular assistive device. FIG. 5 shows the prediction of pre-transplant inotrope therapy, left ventricular assist device, and both clinical factors on posttransplant PGD. Two marker panel PGD predictions: 136 pairwise combinations of the 17 significantly predictive clinical and protein markers were investigated (FIG. 7A). Overall, panels of inotrope therapy and a protein had significantly increased performance than combinations of 2 proteins (AUROC 0.6505±0.02980 vs 0.6070±0.0454 p-value=2.123E-4; FIG. 7B). For combinations involving pre-transplant inotrope therapy, the addition of KLKB1 outperformed all other protein combinations (AUROC 0.7181 [0.7020, 0.7372]). Protein-protein marker panels containing KLKB1 outperformed all panels composed of other protein markers (t-test, p-values: 2.193E-13 to 6.634E-02; FIG. 7C). The best performing panel overall and for each patient cohort was a combination of pre-transplant inotrope therapy and expression of KLKB1 protein (FIG. 7D). There were 52 marker panels significantly more predictive than the most predictive marker KLKB1 on its own.

Tables 6 and 7 describe the biological pathways where proteins expressed in PGD patients were significantly different than in patients without PGD. The difference would be enriched if the expression was higher in PGD patients and depleted if the expression was lower in PGD patients.

TABLE 6
Depleted Functions and Pathways in PGD.

| | Normalized | | False | |
|---|---|---|---|---|
| | Enrichment | | Discovery | |
| Term | Score | P-value | Rate | Category |
| serine-type endopeptidase inhibitor activity | −13.0783 | 0.0305 | 0.0817 | GO_Molecular_Function_2017b |
| (GO:0004867) | | | | |
| Platelet degranulation_Homo sapiens_R-HSA-114608 | −18.2190 | 0.1318 | 0.1783 | Reactome_2016 |
| Response to elevated platelet cytosolic | −19.1522 | 0.1261 | 0.1848 | Reactome_2016 |
| Ca2+_Homo sapiens_R-HSA-76005 | | | | |

TABLE 7
Enriched Pathways and Functions in PGD

| | Normalized | | False | |
|---|---|---|---|---|
| | Enrichment | | Discovery | |
| Term | Score | P-value | Rate | Category |
| Systemic lupus erythematosus | 2.5112 | 0.0004 | 0.0011 | KEGG_2019_Human |
| Complement Activation WP545 | 2.3829 | 0.0028 | 0.0057 | WikiPathways_2019_Human |
| Selenium Micronutrient Network WP15 | 2.1277 | 0.0128 | 0.0136 | WikiPathways_2019_Human |
| Staphylococcus aureus infection | 1.8304 | 0.0456 | 0.0546 | KEGG_2019_Human |
| Regulation of Complement | 2.1148 | 0.0153 | 0.0736 | Reactome_2016 |
| cascade_Homo sapiens_R-HSA-977606 | | | | |
| Complement and coagulation cascades | 1.5689 | 0.0632 | 0.1198 | KEGG_2019_Human |

The panel of inotrope therapy and KLKB1 showed the least variation while maintaining high performance across all cohorts (95% AUROC CI above 0.7; FIG. 7E).

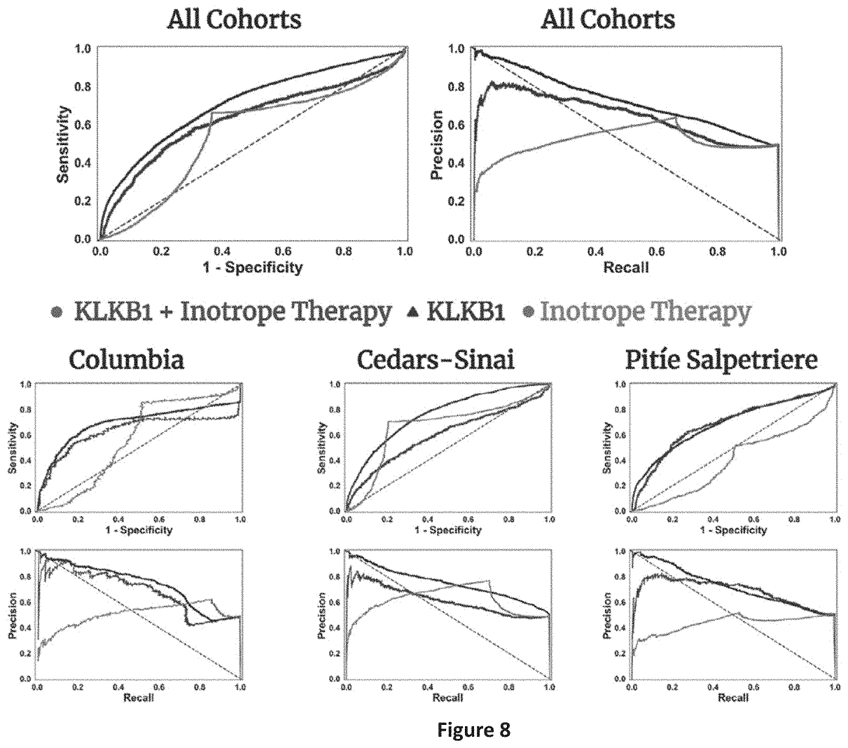

PGD classifier performance: Each panel's predictions form a 2-marker classifier equation, as shown for the KLKB1 protein and inotrope therapy panel in FIG. 7F. The classifier equation for inotrope therapy and KLKB1 is the summation of multiplying −0.9946 by a binary value of pre-transplant inotrope therapy (0 or 1) and −2.140 by normalized pre-transplant KLKB1 expression. This equation demonstrates an inverse relationship between post-transplant PGD risk and either pre-transplant KLKB1 expression or inotrope therapy (or both). The PGD classifier has significantly increased performance compared to the markers on their own (Kolmogorov-Smirnov 2-sample test p-values<2.165E-23; FIG. 8 and Table 8).

TABLE 8
Performance of KLKB1, Intrope Therapy, and Two-Marker Panel
Predictive Performance Across and Within Patient Cohorts.

| | AUROC | | | |
|---|---|---|---|---|
| | KLKB1 + | | | |
| Cohort | Inotrope therapy | KLKB1 | Inotrope therapy | |
| All | 0.7181 [0.7020, 0.7372] | 0.6444 [0.6293, 0.6655] | 0.5618 | [0.5387, 0.5800] |
| Columbia | 0.7125 [0.6680, 0.7571] | 0.6507 [0.5917, 0.6933] | 0.6005 | [0.5505, 0.6574] |
| Cedars-Sinai | 0.7782 [0.7542, 0.7982] | 0.6094 [0.5787, 0.6326] | 0.677 | [0.6532, 0.7114] |
| Pitié-Salpêtrière | 0.6711 [0.6417, 0.7018] | 0.6984 [0.6731, 0.7279] | 0.4048 | [0.3702, 0.4412] |

| | AUPRC | | | |
|---|---|---|---|---|
| | KLKB1 + | | | |
| Cohort | Inotrope therapy | KLKB1 Inotrope | | Inotrope therapy |
| All | 0.7322 [0.7092, 0.7486] | 0.6659 | [0.6434, 0.6968] | 0.5213 [0.4974, 0.5410] |
| Columbia | 0.7547 [0.7232, 0.8048] | 0.724 | [0.6856, 0.7763] | 0.5211 [0.4778, 0.5816] |
| Cedars-Sinai | 0.7754 [0.7425, 0.8035] | 0.6344 | [0.6026, 0.6708] | 0.6262 [0.5938, 0.6637] |
| Pitié-Salpêtrière | 0.7053 [0.6750, 0.7381] | 0.6659 | [0.6557, 0.6708] | 0.4388 [0.4011, 0.4716] |

TABLE 9
Performance comparison between existing PGD predictors and KLKB1 and Inotrope Therapy Two-Marker Panel.

| | AUROC | | | | |
|---|---|---|---|---|---|
| | KLKB1 + | | | | |
| Cohort | Inotrope therapy | CVP/PCWP | MELD | Radial Score | |
| All | 0.7181 [0.7020, 0.7372] | 0.3868 [0.3709, 0.4026] | 0.3759 [0.3615, 0.3922] | 0.3917 | [0.3760, 0.4070] |
| Columbia | 0.7125 [0.6680, 0.7571] | 0.4189 [0.3753, 0.4644] | 0.4175 [0.3832, 0.4586] | 0.4893 | [0.4502, 0.5313] |
| Cedars-Sinai | 0.7782 [0.7542, 0.7982] | 0.3951 [0.3728, 0.4147] | 0.3531 [0.3333, 0.3687] | 0.383 | [0.3546, 0.4017] |
| Pitié-Salpêtrière | 0.6711 [0.6417, 0.7018] | 0.3619 [0.3409, 0.3877] | 0.3847 [0.3648, 0.4053] | 0.3551 | [0.3335, 0.3863] |

AUPRC

| | KLKB1 + | | | |
|---|---|---|---|---|
| Cohort | Inotrope therapy | CVP/PCWP | MELD | Radial Score |
| All | 0.7322 [0.7092, 0.7486] | 0.4266 [0.4034, 0.4439] | 0.4257 [0.4040, 0.4417] | 0.4283 [0.4069, 0.4439] |
| Columbia | 0.7547 [0.7232, 0.8048] | 0.4563 [0.4192, 0.5151] | 0.4411 [0.4037, 0.4870] | 0.4815 [0.4430, 0.5296] |
| Cedars-Sinai | 0.7754 [0.7425, 0.8035] | 0.4282 [0.4041, 0.4569] | 0.4122 [0.3841, 0.4372] | 0.4205 [0.3925, 0.4439] |
| Pitié-Salpêtrière | 0.7053 [0.6750, 0.7381] | 0.4194 [0.3844, 0.4419] | 0.4466 [0.4093, 0.4685] | 0.4197 [0.3831, 0.4431] |

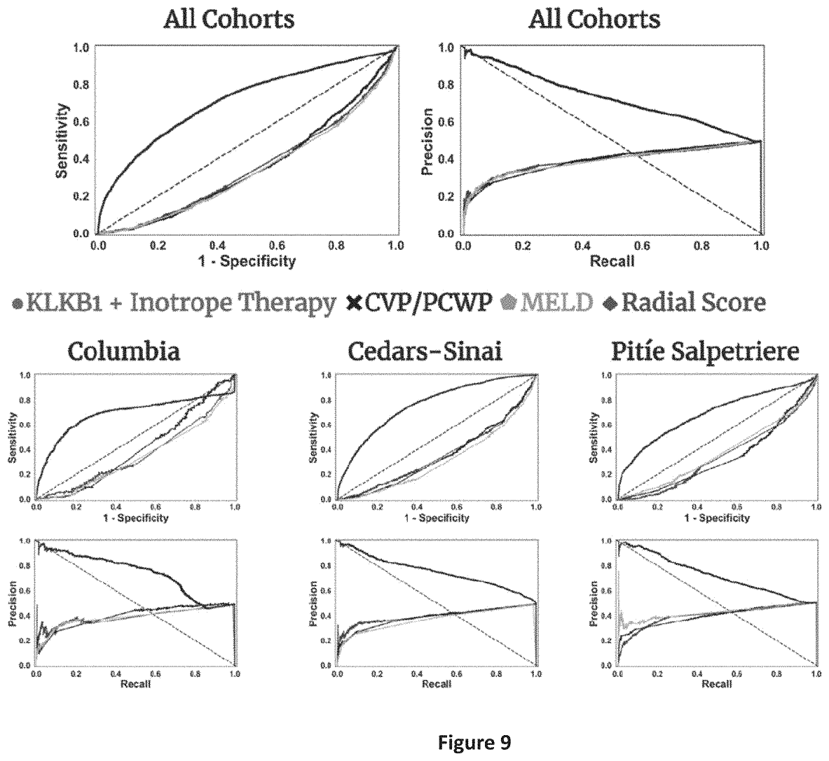

The disclosed prediction panel was compared to existing PGD predictors: the RADIAL score, the MELD score, and the CVP/PCWP ratio. The 2-marker panel significantly outperforms all composite scores by 50% on average (FIG. 9; Kilogorov Smirnov 2-sample p-values<2.165E-23).

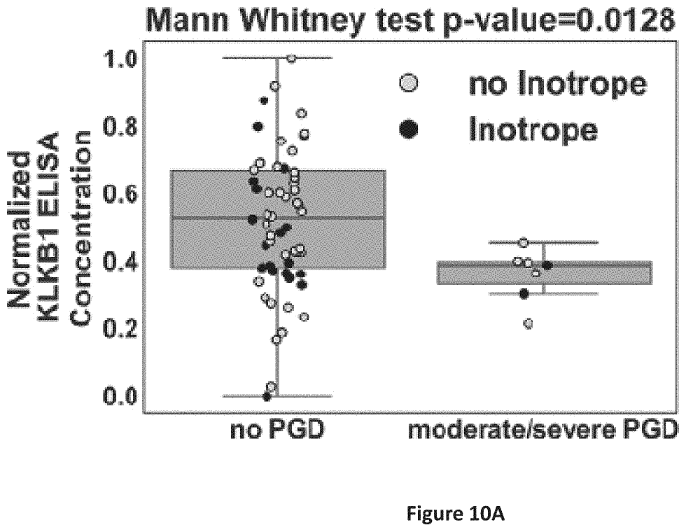

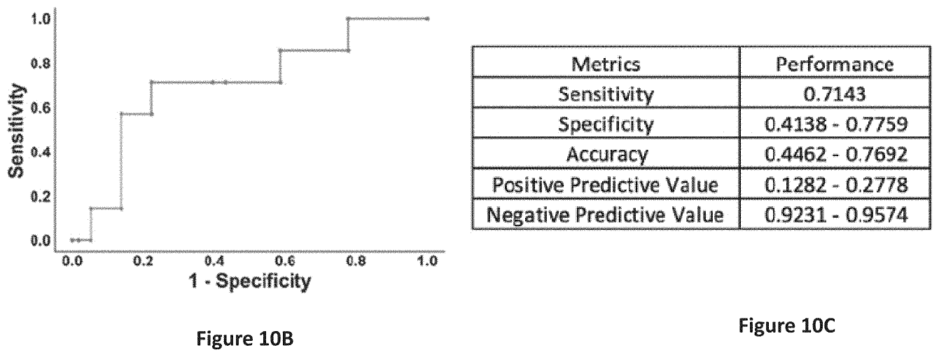

Whole serum KLKB1 ELISA in PGD: a validation cohort of 65 consecutive patients' serum samples was prospectively collected on the day prior to a heart transplant at CUIMC. Whole serum was used for KLKB1 ELISA to test the feasibility of a clinical test without microvesicle purification. Patients who had RV PGD or mechanical support for reasons other than PGD were excluded from the analysis. Potentially due to the small number of severe PGD (n=3), there was no significant difference in average KLKB1 levels when comparing patients with severe PGD to no PGD levels (Mann-Whitney U test 19.81±6.248 vs 45.796±32.54 p-value=0.0511). However, by adding patients with moderate PGD (n=4), defined per ISHLT guidelines as moderate LV dysfunction requiring pharmacologic but not mechanical support, KLKB1 levels were significantly lower (Mann-Whitney U test 20.44±11.40 vs 45.796±32.54 015 p-value=0.0128; FIG. 10A). The putative PGD classifier from the original proteomic data produces an AUROC of 0.7143 to predict moderate/severe PGD compared to patients who did not have PGD (FIG. 10B). The incidence of PGD in this cohort more closely approximates the national PGD rate of 7.4%, and in this setting, the classifier was marked by a high sensitivity and negative predictive value but a low specificity and positive predictive value (FIG. 10C and Table 10).

TABLE 10
Two marker panel equation performance on validation data. True Positive
TP; True Negative TN; False Positive FP; False Negative FN; Positive Predictive
Value PPV; Negative Predictive Value NPV.

| Threshold | TP | FP | FN | TN | Sensitivity | Specificity | PPV | NPV |
|---|---|---|---|---|---|---|---|---|
| 0.0538 | 7 | 58 | 0 | 0 | 1 | 1 | 0.1077 | |
| 0.1389 | 7 | 45 | 0 | 13 | 1 | 0.7759 | 0.1346 | 1 |
| 0.1394 | 6 | 45 | 1 | 13 | 0.8571 | 0.7759 | 0.1176 | 0.9286 |
| 0.1622 | 6 | 34 | 1 | 24 | 0.8571 | 0.5862 | 0.15 | 0.96 |
| 0.1658 | 5 | 34 | 2 | 24 | 0.7143 | 0.5862 | 0.1282 | 0.9231 |
| 0.2167 | 5 | 25 | 2 | 33 | 0.7143 | 0.4310 | 0.1667 | 0.9429 |
| 0.2210 | 5 | 23 | 2 | 35 | 0.7143 | 0.3966 | 0.1786 | 0.9459 |
| 0.2752 | 5 | 13 | 2 | 45 | 0.7143 | 0.2241 | 0.2778 | 0.9574 |
| 0.2821 | 4 | 13 | 3 | 45 | 0.5714 | 0.2241 | 0.2353 | 0.9375 |
| 0.2984 | 4 | 8 | 3 | 50 | 0.5714 | 0.1379 | 0.3333 | 0.9434 |
| 0.3271 | 1 | 8 | 6 | 50 | 0.1429 | 0.1379 | 0.1111 | 0.8929 |
| 0.3883 | 1 | 3 | 6 | 55 | 0.1429 | 0.0517 | 0.25 | 0.9016 |
| 0.4013 | 0 | 3 | 7 | 55 | 0 | 0.0517 | 0 | 0.8871 |
| 0.4852 | 0 | 1 | 7 | 57 | 0 | 0.0172 | 0 | 0.8906 |
| 1.4852 | 0 | 0 | 7 | 58 | 0 | 0 | NA | 0.8923 |

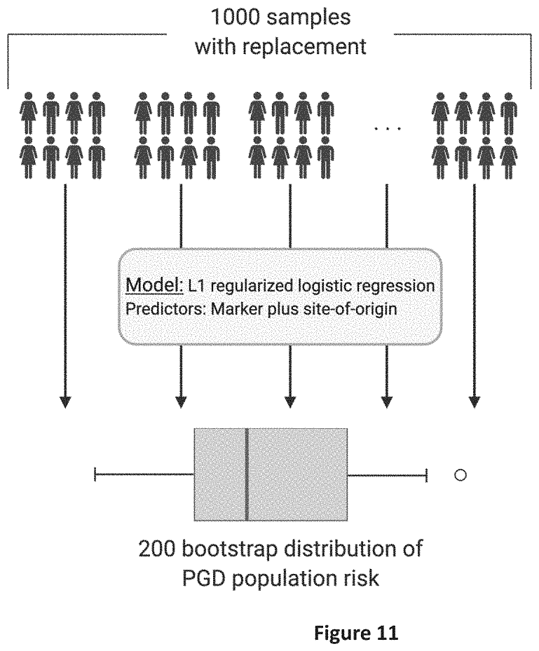

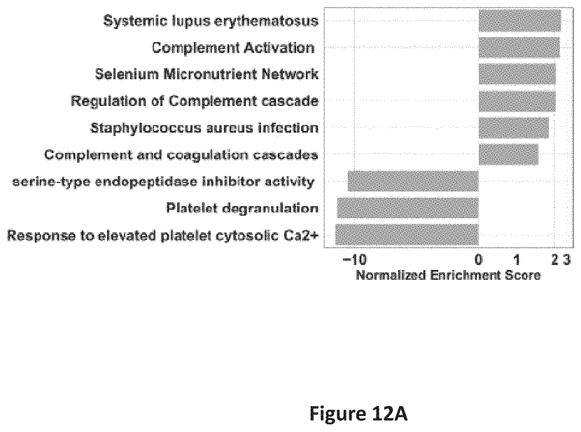

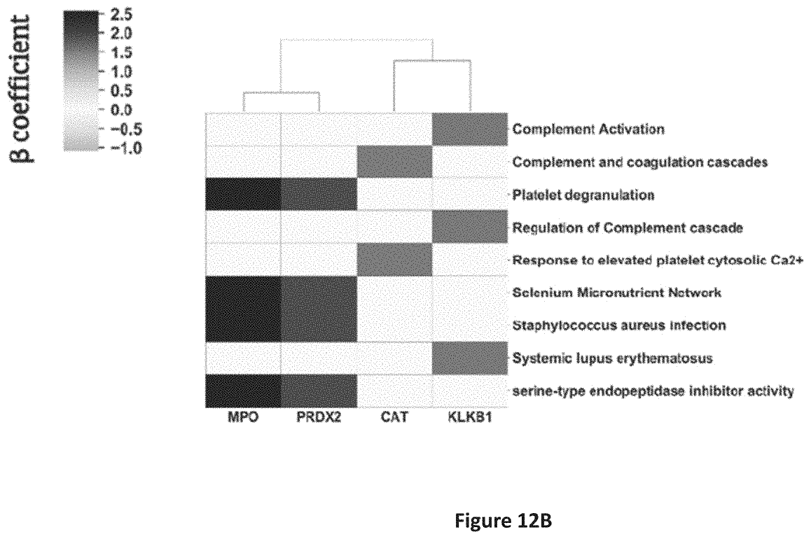

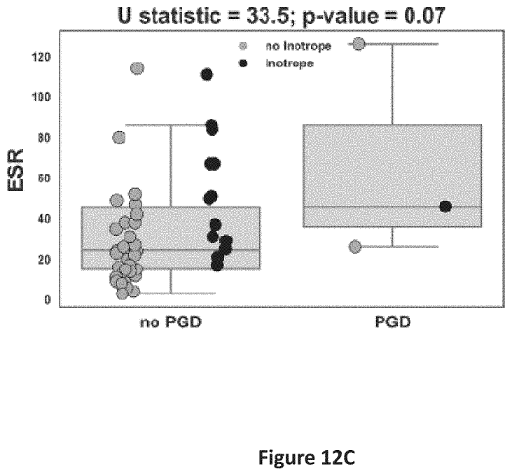

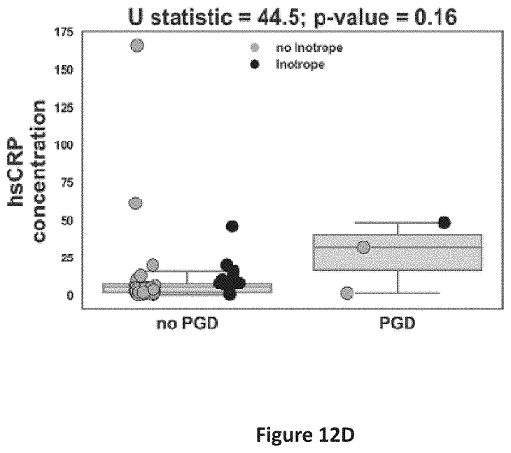

Primary graft dysfunction pathway analysis and clinical tests in patients: to investigate PGD pathogenesis, a differential expression signature was calculated from proteomic data (262 proteins, including immunoglobulins, identified in all patients with corresponding gene names) (FIG. 11). FIG. 11 shows the differential protein analysis modeling scheme. The post-transplant PGD population risk of each marker is shown. From the 88 patients, sampling with replacement (e.g., over- and under-representing males and females in a population as shown here) was performed prior to the fitting model. The fitted model can estimate the population risk towards PGD occurrence of a marker controlling for the patient's site of origin. The sampling was random sampling and fit the model 200 times. The 200 bootstrap distribution produces a confidence interval for PGD population risk for each marker. The average of each distribution is the population risk value for that marker. In the case of using protein markers, the collection of the population risk values is the differential expression signature towards primary graft dysfunction. Gene set enrichment analysis (GSEA) was used to investigate enriched pathways and functions from the differential protein signature. 6 pathways were significantly enriched (Table 11; FDR<0.2), and 3 pathways were depleted in patients with PGD (Table 12; FDR<0.2; FIG. 12A).

TABLE 11
Enriched Pathways and Functions in PGD.

| | | Normalized | | | False | |
|---|---|---|---|---|---|---|
| | | Enrichment | | | Discovery | |
| ID | Term | Score | Ledge Genes | P-value | Rate | Category |
| 05322 | Systemic lupus | 2.5112 | HIST1H4A; HIST1H3A; C4B; CIQC; | 0.0004 | 0.0011 | KEGG_2019_ |
| | erythematosus | | C8G; C1QB; C8A; C5; C1S; C7; C9; | | | Human |
| | | | C1QA; C1R; C8B; C6 | | | |
| WP545 | Complement | 2.3829 | C4B; C1QC; C8G; C1QB; C8A; C5; | 0.0028 | 0.0057 | WikiPathways_ |
| | Activation | | MASP2; C1S; C7; CFP; C9; C1QA; | | | 2019_Human |
| | | | C1R; C8B; C6 | | | |
| WP15 | Selenium | 2.1277 | PRDX2; MPO; CAT; HBB; HBA1 | 0.0128 | 0.0136 | WikiPathways_ |
| | Micronutrient | | | | | 2019_Human |
| | Network | | | | | |
| 05150 | Staphylococcus | 1.8304 | DEFA1; CFH; C4B; C1QC; CFI; CAMP; | 0.0456 | 0.0546 | KEGG_2019_ |
| | aureus infection | | C1QB; C5; MASP2; C1S; C1QA; C1R | | | Human |
| R-HSA- | Regulation of | 2.1148 | CFH; C4B; CFI; C8G; C8A; C5; C7; | 0.0153 | 0.0736 | Reactome_2016 |
| 977606 | Complement | | C4BPB; C9; C8B; PROS1; C6; CFHR3; | | | |
| | cascade | | C4BPA; C3 | | | |
| 04610 | Complement | 1.5689 | CFH; C4B; C1QC; CFI; F5; C&G; F13A1; | 0.0632 | 0.1198 | KEGG_2019_ |
| | and coagulation | | C1QB; F13B; C8A; C5; CPB2; MASP2; | | | Human |
| | cascades | | C1S; C7; C4BPB; C9; C1QA; C1R; C8B; | | | |
| | | | PROS1; F11; C6; KNG1; F10; C4BPA; | | | |
| | | | C3; PROC; PLG; FGA; F9; SERPINA5; | | | |
| | | | FGG; SERPINA1; VWF; FGB; C2; CFD; | | | |
| | | | MBL2; F12; SERPINF2 | | | |

The sets of proteins involved within each pathway and function in combination were evaluated to predict PGD in patients. The same MCCV methodology and the prediction significance thresholds defined above were used for this analysis. Out of 196 proteins, 8 proteins were found to be significantly predictive within at least 1 of the 136 pathways and functions: KLKB1, PRDX2, TPM4, MPO, CAT, HSPA5, IGHD and IGLV2-11 (Table 12). Significant protein predictions within these pathways and functions (FIG. 12B) revealed enrichment of processes related to inflammation, coagulation, and activation of the innate immune system. Downregulation of KLKB1 was identified in the activated complement and immune response pathways.

TABLE 12
Depleted Functions and Pathways in PGD.

| | | Normalized | | | False | |
|---|---|---|---|---|---|---|
| | | Enrichment | | | Discovery | |
| ID | Term | Score | Ledge Genes | P-value | Rate | Category |
| GO:000 | serine-type | −13.0783 | AHSG; | 0.0305 | 0.0817 | GO_Molecular_ |
| 4867 | endopeptidase | | SERPINA10; | | | Function_ |
| | inhibitor | | PROS1; AGT; | | | 2017b |
| | activity | | SERPINF1; | | | |
| | | | SERPINA5; | | | |
| | | | SERPINA1; | | | |
| | | | SERPINF2; AMBP; | | | |
| | | | SERPINA4; | | | |
| | | | SERPINA6; ITIH4; | | | |
| | | | LPA; SERPINA7; | | | |
| | | | SERPIND1; ITIH3; | | | |
| | | | SERPINA3; HRG; | | | |
| | | | SERPINC1; A2M; | | | |
| | | | PZP; ITIH2 | | | |
| R-HSA- | Platelet | −18.2190 | ECM1; PLG; FGA; | 0.1318 | 0.1783 | Reactome_ |
| 114608 | degranulation | | LGALS3BP; FGG; | | | 2016 |
| | | | VCL; PF4; | | | |
| | | | SERPINA1; VWF; | | | |
| | | | THBS1; FGB; CFD; | | | |
| | | | SERPINF2; | | | |
| | | | SERPINA4; A1BG; | | | |
| | | | QSOX1; ITIH4; | | | |
| | | | IGF2; APOA1; | | | |
| | | | APOH; TF; ITIH3; | | | |
| | | | SERPINA3; HRG; | | | |
| | | | PPBP; CLU; A2M; | | | |
| | | | CLEC3B; ALB | | | |
| R-HSA- | Response to | −19.1522 | ECM1; PLG; FGA; | 0.1261 | 0.1848 | Reactome_ |
| 76005 | elevated | | LGALS3BP; FGG; | | | 2016 |
| | platelet | | VCL; PF4; | | | |
| | cytosolic Ca2+ | | SERPINA1; VWF; | | | |
| | | | THBS1; FGB; CFD; | | | |
| | | | SERPINF2; | | | |
| | | | SERPINA4; A1BG; | | | |
| | | | QSOX1; ITIH4; | | | |
| | | | IGF2; APOA1; | | | |
| | | | APOH; TF; ITIH3; | | | |
| | | | SERPINA3; HRG; | | | |
| | | | PPBP; CLU; A2M; | | | |
| | | | CLEC3B; ALB | | | |
| | | | ECM1; PLG; FGA; | | | |
| | | | LGALS3BP; FGG; | | | |
| | | | VCL; PF4; | | | |
| | | | SERPINA1; VWF; | | | |
| | | | THBS1; FGB; CFD; | | | |
| | | | SERPINF2; | | | |
| | | | SERPINA4; A1BG; | | | |
| | | | QSOX1; ITIH4; | | | |
| | | | IGF2; APOA1; | | | |
| | | | APOH; TF; ITIH3; | | | |
| | | | SERPINA3; HRG; | | | |
| | | | PPBP; CLU; A2M; | | | |
| | | | CLEC3B; ALB | | | |

Markers of inflammation were also analyzed in the validation cohort. There was a trend towards increased erythrocyte sedimentation rate (66.0±43.20 vs. 33.70±26.86 Mann-Whitney U test p-value=0.07; FIG. 12C). Protein (27.24±19.51 vs 11.27±25.54, p-value=0.16; FIG. 12D) and complement levels were not significantly altered (FIG. 2). This analysis was hampered by a small number of severe PGD patients and wide confidence intervals. However, there does appear to be some laboratory trend towards increased inflammation corresponding with the results of the GSEA analysis.

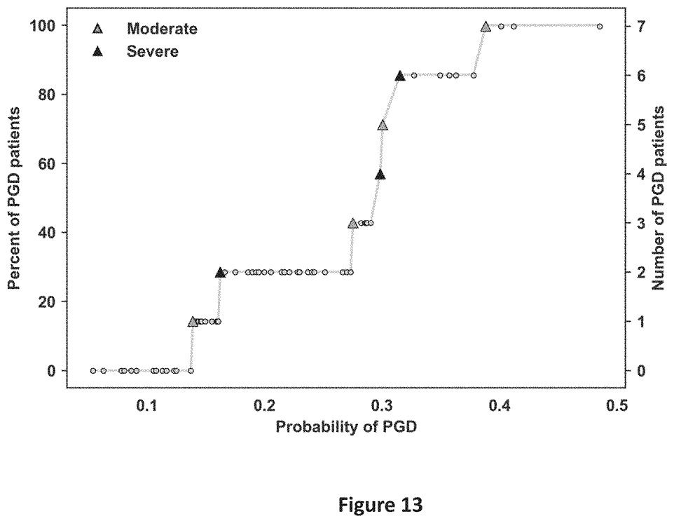

FIG. 13 shows a calibration curve for PGD prediction by a putative classifier on 80 CUIMC patient assessment data. Probabilities of PGD risk versus Percent/Number of PGD patients of the CUIMC assessment patients are shown in FIG. 13. Patients who had moderate or severe PGD are shown as enlarged triangles, and those who did not are circles on the calibration curve. The probabilities calculated are the logit-transformed dot product between the assessment data (KLKB1 ELISA expression and pre-transplant Inotrope therapy) and the putative PGD classifier.

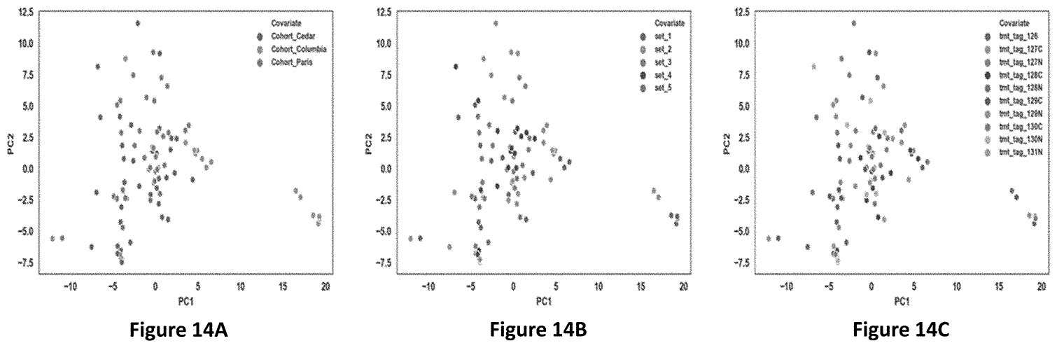

FIGS. 14A-14C show principal components of protein expression and association with covariates. Overlay of Site-of-origin (FIG. 14A), Set (FIG. 14B), and TMT-Tag covariates on protein expression variation (FIG. 14C), determined via Principal Components Analysis for patients are shown. Set/TMT Tag (or experimental batch) is accounted for during protein identification and quantification, while each patient cohort was a different experiment and not accounted for during this process. As shown in the principal components analysis, cohort site of origin explains protein expression variation for patients and thus is included as a covariate in association and prediction analyses. PCA can determine the most variability found with the protein expression data, where this variability can come from non-biological variability. Therefore patients were projected onto their variability components to assess which non-biological variability explains the observed differences in the protein expression data.

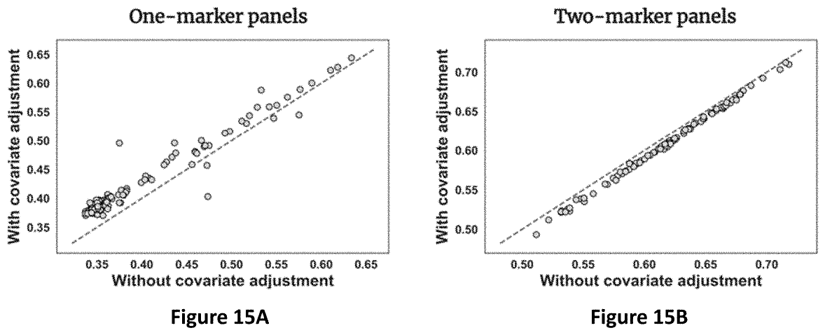

FIGS. 15A-15B show correlation between unadjusted and adjusted individual and two marker panel performances. Comparison between model specifications for the individual (FIG. 15A) and two marker panel predictions (FIG. 15B) when including and not including covariate adjustment (i.e., cohort site-of-origin) are shown. The marker prediction specifications did not include site-of-origin as covariates in order to easily translate the putative classifier equations to new patient data. However, when covariate adjustment was included, the average AUROC performance is highly correlated with the unadjusted performances suggesting minimal confounding by site and accuracy of the classifier equations to translate onto new patient data agnostic of site. This analysis was performed to generate evidence in using simpler and more interpretable machine learning models that did not account for patient site of origin.

Pre-heart transplant recipient clinical and proteomic markers predictive of post-transplant PGD were identified using a data-driven methodology to generate a clinically interpretable PGD classifier. Machine learning and statistical techniques were used to mitigate confounding in biological enrichment analyses and improve predictive accuracy with modest population size. Reduction in KLKB1 was the strongest predictor of PGD both by itself and in combination with other markers. KLKB1 is a serine protease that controls the activation of both inflammation and coagulation in what is known as the kallikrein-kinin-system (KKS). In the inflammatory response, KLKB1 converts high molecular weight kininogen into bradykinin, stimulating the release of nitric oxide and prostacyclin, causing vasodilation and increased vascular permeability. It also acts as a neutrophil chemoattractant, causing degranulation. Evaluations of the KKS system in patients with sepsis, a markedly inflammatory state, demonstrated increased KKS activity, characterized by decreased levels of plasma kallikrein, likely due to consumption. Decreases in KLKB1 have been noted in typhoid fever, ARDS, cardiopulmonary bypass and in normal volunteers infused with gram-negative endotoxin. Similarly, in animal models of inflammatory bowel disease and inflammatory arthritis, plasma kallikrein levels were markedly reduced.

Other predictive proteins identified were likewise involved in either inflammation or innate immunity, including PRDX2, MPO, PGLYRP2, and DEFA1. Similarly, enrichment analysis of protein expression differences demonstrated several upregulated biological processes, including inflammatory and immune pathways in patients prior to PGD. Laboratory tests in the validation cohort trended towards increased inflammation though were not significant. It remains to be seen whether this inflammatory signature is purely a bio-marker or contributes to PGD and, importantly, whether modifying this state can have an impact on the evolution of PGD.

The lack of inotrope therapy was predictive of PGD, and this stands in contrast to prior analyses, which demonstrated that the presence of inotrope therapy was associated with PGD. Pre-transplant inotrope therapy and durable mechanical support (such as LVAD) were exclusive prior to transplant, and mechanical support has been associated with PGD in prior studies. However, mechanical support was not significantly predictive of PGD in the analyses and did not interact with inotrope therapy in prediction models. Whether inotrope therapy itself is an actual driver of PGD protection versus an epiphenomenal marker remains to be explored. There were clear differences in medical therapy, anticoagulation and mechanical support between patients receiving and not receiving inotrope therapy (Table 13).

TABLE 13
Clinical characteristic population associations to PGD

| | Clinical Characteristic | | | | |
|---|---|---|---|---|---|
| | | Odds | | Odds | |
| | | Lower | Odds | Upper | Significant |
| | Age | Bound | Mean | Bound | (*) |
| Patient characteristics | BMI | 1 | 1.3967 | 4.1971 | |
| | Blood Type | 0.9997 | 1.6386 | 6.3245 | |
| | A | | | | |
| | AB | 0.331 | 0.776 | 1 | |
| | B | 0.7134 | 1.0389 | 1.5964 | |
| | O | 0.4463 | 0.9835 | 1.4867 | |
| | Donor Age | 0.9992 | 1.948 | 5.7177 | |
| | Sex = F | 0.9968 | 1.2897 | 3.5424 | |
| | History of Tobacco Use = Y | 0.6979 | 1.3704 | 3.4798 | |
| | Diabetes = Y | 0.4639 | 1.0347 | 2.0246 | |
| | Ischemic = Y | 0.999 | 1.5667 | 3.9011 | |
| Cardiomyopathy | Non-Ischemic | 0.5969 | 1.0569 | 2.0048 | |
| | Adriamycin | | | | |
| | Amyloid | 1 | 1.1953 | 3.4071 | |
| | Chagas | 0.4088 | 0.9495 | 1 | |
| | Congenital | 1 | 1.0393 | 1.1444 | |
| | Hypertrophic | 1 | 1.0218 | 1.0011 | |
| | Idiopathic | 0.5467 | 0.972 | 1 | |
| | Myocarditis | 0.3377 | 0.8321 | 1.274 | |
| | Valvular Heart Disease | 1 | 1.0143 | 1 | |
| | Viral | 1 | 1.0096 | 1 | |
| | Ischemic Time | 0.661 | 0.9799 | 1 | |
| Transplant factors | Ventricular Assist Device = Y | 1 | 2.6219 | 10.2286 | |
| | PA Diastolic | 0.7261 | 1.3762 | 3.627 | |
| Hemodynamics | PA Systolic | 0.8087 | 0.9847 | 1 | |
| | PA Mean | 1 | 1.1289 | 2.8667 | |
| | CVP | 1 | 1.4048 | 4.6097 | |
| | PCWP | 1 | 1.1958 | 2.7944 | |
| | Creatinine | 0.7393 | 0.9934 | 1 | |
| Lab values | INR | 1 | 1 | 1 | |
| | TBILI | 1 | 1.1847 | 2.533 | |
| | Sodium | 0.1139 | 0.6812 | 1 | |
| | Antiarrhythmic = Y | 0.0429 | 0.3604 | 1 | |
| Medications | Beta Blocker = Y | 1 | 1.8671 | 4.8259 | |
| | Inotrope = Y | 0.8432 | 1.5655 | 3.6569 | |
| | CVP/PCWP | 0.0916 | 0.3239 | 0.7983 | * |
| Composite Scores | MELD | 1 | 1.9121 | 8.1395 | |
| | RADIAL Score | 0.6234 | 0.9946 | 1.1056 | |

Integrating both proteomic and clinical variables into one model demonstrated that combinations of proteins and clinical characteristics can yield increased classification power. KLKB1 combinations resulted in the greatest classification performance. Interestingly, though inotrope therapy alone demonstrated modest prediction, its combination with KLKB1 resulted in the greatest increase in classification power when compared to the combination of KLKB1 and other top-performing proteins. Notably, this panel outperforms other composite scores and clinical variables such as the RADIAL score, which demonstrated low performance in all three cohorts.

Whether the proteomic results were being driven by a specific microvesicular process or a reflection of the greater overall serum milieu was tested in the validation ELISA cohort. The ELISA samples themselves were not able to generate a classifier using KLKB1 and inotrope therapy due to the paucity of PGD samples in that cohort. However, the proteomics-derived classifier generated a similar AUROC on whole serum as it did in the original microvesicle proteomic cohort. At the whole serum level, in a population whose incidence mirrored closely to national PGD rates, the classifier performed essentially as a rule-out test with a very high negative predictive value.

The disclosed classifier performed well when absolute values of KLKB1 in the serum were normalized by ELISA. With only 3 cases of severe PGD in this cohort, which approximates the normal incidence of PGD, KLKB1 trended towards a significant decrease in PGD patients (p=0.051). Looking forward to clinical utility, PGD risk stratification can be served in the outpatient setting as part of an overall pre-transplant evaluation. The disclosed subject matter can be used for understanding if the patient risk is static or evolves and whether changes in that risk were associated with clinical status. The optimistic potential here is to use this classifier to evaluate therapies that can alter future PGD risk and improve heart transplant outcomes.

### Example 2: Alterations in the Kallikrein-Kinin System Predict Death after Heart Transplant

Methods

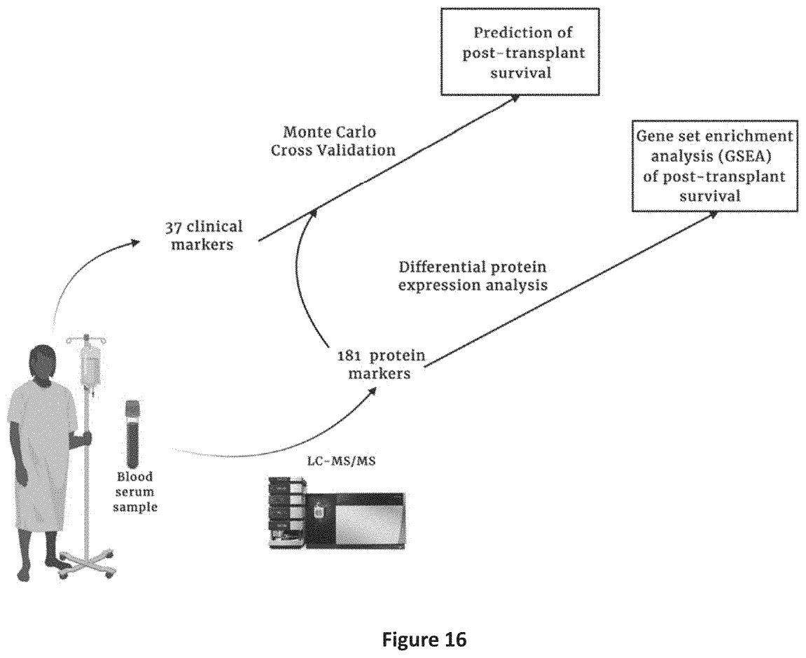

Patient cohorts. A study overview is provided in FIG. 16. The study was designed in accordance with the rules of Good Clinical Practice and with the ethical principles established in the Declaration of Helsinki. The cohort of patients used in this study was previously described in Giangreco et al. (J. Hear. Lung Transplant, 2021) comprising heart transplant patients with and without severe primary graft dysfunction (PGD) using ISHLT criteria matched by gender and age. Patient serum samples were prospectively recruited at Columbia University Irving Medical Center (Columbia) between 2014 and 2016. Patient serum samples were retrospectively collected from biobanks at Cedars-Sinai hospital (Cedars) and Pitié Salpêtrière University Hospital (Paris). Patients undergoing re-transplant were excluded. For 81 patients, a single serum sample was provided and analyzed. Seven patients from the Paris cohort had two serum samples provided and all expression and prediction analyses averaged the protein quantities of those two samples. Human subjects protocol was approved by the Institutional Review Boards of Columbia University, Cedars Sinai and Pitié Salpêtrière University Hospital and patients provided informed consent. Patient characteristics were collected including demographics, biometrics, labs, medications and hemodynamics. The MELD-XI score was derived for each patient using the formula:

3.78×ln[serum bilirubin (mg/dL)]+9.57×ln[serum creatinine (mg/dL)]+6.43 (2)

A multivariate logistic regression model was performed to determine significance of each clinical characteristic's association to patient survival amongst all clinical characteristics. For characteristics missing in less than a third of patients, the most frequent value or the average value was imputed for binary/categorical and numeric characteristics respectively. The patient cohort table was constructed using custom Python and R scripts using the tableone R package.

Mass spectrometry analysis. Total microvesicle was isolated from 100 μl of serum using an optimized protocol based on a commercial total microvesicle isolation kit from Life Technologies Inc. (ThermoFisher Total Exosome Isolation from Serum, 4478360), specifically including an incubation at 4 degrees (3) and a resuspension volume of 25 μl (6). Samples were homogenized using MS-compatible lysis buffer (4 M Urea/50 mM Ammonium bicarbonate/protease inhibitor & phosphatase inhibitor). 20 μg of lysate from each sample was proteolytically cleaved with trypsin and chemically labeled with mass spectrometer detectable quantification reagent, TMT10plex isobaric mass tags separately. Sample preparation quality control was performed by TMT labels checking and tryptic digestion efficiency (100 ng of each sample was pooled, desalted, and analyzed by short SPS-MS3 method, and using normalization factor, samples were bulk mixed at 1:1 across all channels). Quality control to check LC-MS performance was performed using Pierce™ HeLa Digest/PRTC Standard (Catalog number: A47997) and Pierce™ TMT11plex Yeast Digest Standard (Catalog number: A40938).

A reference sample was generated by pooling equal amounts of serum microvesicles from each patient to create a common protein library for quantification. Samples were bulk mixed at 1:1 across all channels and bulk mixed samples were fractionated using the Pierce™ High pH Reversed-Phase Peptide Fractionation Kit (Thermo Scientific). Each fraction was dried down in a speed-vac and dissolved in a solution of 2% acetonitrile/2% formic acid. Each fraction was injected in triplicate on Oribitrap Fusion coupled with the UltiMate™ 3000 RSLCnano system (Thermo Scientific). Fractionated peptides were separated from the self-made 25 cm column (Resprosil-C18, 2.4 mm, 25 cm×75 mm, Dr. Maisch GmbH) at a non-linear flow rate of 300 nl/min using a gradient of 5-30% of buffer B (0.1% (v/v) formic acid, 100% acetonitrile) for 70 min with a temperature of the column maintained at 40° C. during the entire experiment. The full MS spectra were acquired in the Orbitrap Fusion™ Tribrid™ Mass Spectrometer (Thermo Scientific) at a resolution of 120,000. The 10 most intense MS1 ions were selected for MS2 analysis. The isolation width was set at 0.7 Da and isolated precursors were fragmented by CID at a normalized collision energy (NCE) of 35% and analyzed in the ion trap using “turbo” scan speed.

Following acquisition of each MS2 spectrum, a synchronous precursor selection (SPS) MS3 scan was collected on the top 10 most intense ions in the MS2 spectrum. SPS-MS3 precursors were fragmented by higher energy collisioninduced dissociation (HCD) at an NCE of 60% and analyzed using the Orbitrap. Raw mass spectrometric data were analyzed using Proteome Discoverer 2.2 to perform database search and TMT reporter ions quantification. TMT tags on lysine residues and peptide N termini (+229.163 Da) and the carbamidomethylation of cysteine residues (+57.021 Da) was set as static modifications, while the oxidation of methionine residues (+15.995 Da), deamidation (+0.984) on asparagine and glutamine were set as a variable modification. Data were searched against a UniProt human database with peptide-spectrum match (PSMs) and protein-level at 1% FDR. The signal-to-noise (SN) measurements of each protein were normalized so that the sum of the signal for all proteins in each channel was equivalent to account for equal protein loading. The results obtained from PD2.2 were further analyzed as described below.

Protein expression analysis. A differential protein expression signature was calculated between survived and expired patient samples, as previously described in Giangreco et al. (J. Hear. Lung Transplant, 2021). The protein association calculated was used as the differential rank statistic for pathway analysis using gene set enrichment analysis (GSEA).

All the statistical analyses were done in the Python programming language (Python Software Foundation. Python Language Reference, version 3.7. The software platform STRING investigated cellular component enrichment of the identified proteins.

The difference in protein expression distributions between the prospective and retrospective cohorts was tested with the Kolmogorov—Smirnov 2-sample test. The protein expression distribution deviation from normality test is from D'Agostino's and Pearson's test, where normality of a distribution is rejected at an alpha level p-value of 0.05. Both methods were from the python package Scipy. A differential protein expression signature was calculated between survived and expired patient samples. To estimate association of individual protein levels to survival, L1-regularized logistic regression models were calculated for each protein with the sites-of-origin as covariates. Two hundred (200) bootstraps (samples with replacement) of the models were performed to determine a confidence interval for the protein expression association to survival. The average of the bootstrap distribution for each protein was used as the differential rank statistic.

For 81 patients, a single serum sample was provided and analyzed. Seven patients from the Paris cohort had two serum samples provided, resulting in 95 total samples. Next, it was examined whether the additional samples were more correlated in the expression of the 181 proteins. Thus, 95 choose 2 or 4465 pairwise (spearman) correlations were computed across 181 proteins. Only 71 (1.6%) had a spearman correlation over 0.5, where 13 included a technical replicate. The variability in sample expression suggests technical replicates were not likely to inflate protein expression differences for patient survival. For the analysis, protein values between the two replicates of the 7 samples were averaged resulting in one sample for each of the 88 patients for downstream analysis.

Pathway analysis was conducted using gene set enrichment analysis (GSEA). The GSEA algorithm employed was from the python package gseapy version 0.9.15. The pathway and function gene lists used in the GSEA analysis were ‘GO_Biological_Process_2017b’, ‘GO_Molecular_Function_2017b’, ‘GO_Cellular_Component_2017b’, ‘Reactome_2016’, ‘WikiPathways_2019_Human’, ‘KEGG_2019_Human’, which were all in the gseapy package hosted on its website. The statistics generated by the GSEA algorithm is detailed in their online user guide. Briefly, the Normalized Enrichment Score (NES) provides a gene set enrichment compared to all permutations of the gene set enrichments for the protein expression data. The NES can be interpreted as the gene set enrichment score corrected for the size of the gene set and spurious, un-interesting correlations between the gene sets and the expression dataset. The p-value estimates the probability of seeing an enrichment score as high or higher among the permutation distribution, and the false discovery rate (FDR) estimates the probability that an enrichment score with a given NES is a false positive finding. The leading edge (ledge) genes were the genes from the pathway gene set with the highest impact on the signal generated for the biological process.

Survival prediction. The prediction scheme, Monte Carlo Cross Validation (MCCV), is comprised of the following procedures repeated 200 times:
(1) Split the data into 85% training and 15% validation sets.
(2) Separately normalize, or subtract the sample mean and divide by the sample standard deviation, the training and testing data.
(3) Using only the sampled training data, compute tenfold cross validation and choose the top performing model parameters for predicting survival status.
(4) Refit the training dataset using the top-prediction model parameters determined in 3.
(5) Predict the survival status of the patients in the yet-to-be-seen validation set using the refit model calculated in 4.

Specifically, 200 randomized training/validation data splits for the prediction procedures outlined above (1) were first computed. Next normalization (2; min—max scaling was performed within the training and validation sets, separately) on the clinical and proteomic data separately for the training and validation data. Within each of the 200 randomized training/validation data splits, a tenfold cross validation (within the training set only) was used to optimize model parameters and perform feature selection (3). Using the chosen parameters and features, the entire training set (4) was trained and this model used to predict survival status on the validation set (5). The survival prediction probabilities were compared to the true survival status to compute the area under the receiver operating characteristic curve (AUROC), and other metrics. The AUROC values reported in this paper were calculated using the validation set patient probabilities. Bootstrapping analysis on the validation patient probabilities (N=50 samples with replacement) resulted in a population distribution for prediction performances, and feature importance (beta coefficient) was extracted within each bootstrap before prediction on the validation set.

A permutation analysis was similarly performed, with random labeling of survival status in patients, to generate and test from a distribution of prediction metrics from random survival assignment. Comparison of the bootstrap and permutation prediction distributions allows for prediction and feature importance comparisons between real and randomly distributed data while accounting for over-fitting during these prediction tasks. The significance of each marker to predict patient survival was evaluated by comparing the 200 feature importance values from the bootstrap and the permutation prediction distributions. The p-values generated in this comparison represent protein marker prediction in the cohort compared with random patient survival. Differences in the bootstrap and permutation distributions were tested using the 2-sample Kolmogorov-Smirnov test.

This methodology permits prediction of death as well as survival. In this case, the machine learning models produce higher probabilities for expired patients which This MCCV methodology samples these patient probabilities to derive an AUROC performance metric and confidence interval. The calculated marker performances were representative of the model's confidence in predicting patient survival.

Several binary schemes were performed to evaluate the predictive results obtained. The main analysis included the binary prediction of post-transplant survival where the patient did not die after transplantation (all-time survival). Covariates were included in the logistic regression model, such as site-of-origin and post-transplant PGD indicators. Finally, post-transplant survival within 1-year were predicted, where patients were labelled as survived as long as they did not die within 1 year of heart transplantation.

Results

1. Patient Clinical Characteristics:

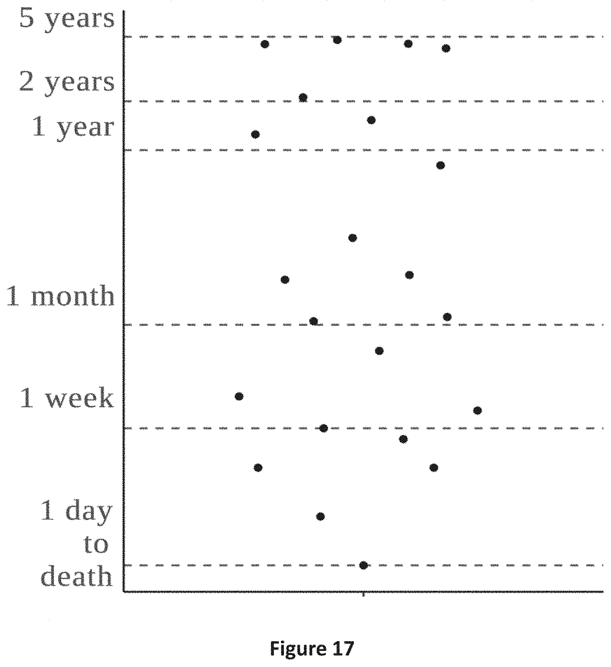

The patient cohort in this study was comprised of 88 patients who underwent heart transplantation between 2014 and 2016 at Cedars Sinai Medical Center (n=43), Pitié Salpêtrière University Hospital (n=29) and Columbia University Irving Medical Center (n=16) (Table 14 and Table 15). There were 37 different pre-transplant clinical characteristics across all the patients including survival post-transplant (Table 14). There were 22 deaths (25%), and a maximum follow up of up to ten years (median: 6.5 years) in this cohort (FIG. 17). No pre-transplant characteristic significantly associated with patient survival were observed (significance alpha threshold of p-value=0.05). In a control analysis, primary graft dysfunction (PGD) after transplant was found to significantly associate with patient survival in both univariate and multivariate analyses (logistic regression p-value=0.019).

2. Microvesicle Proteomics:

Microvesicles were isolated from pre-transplant serum samples and underwent mass spectrometry analysis in at least triplicate per patient (total 322 spectra). Protein expression from each site of collection displayed a non-parametric distribution (Omnibus test of normality p-values<0.001; FIG. 4). Protein expression was significantly different between each site of collection (Columbia comparison to Cedars, Kolmogorov Smirnov test p-value<1.19E-07; Columbia to Paris Kolmogorov Smirnov test p-value=2.38E-05; Paris to Cedars, Kolmogorov Smirnov test p-value=0.008). Of the 681 unique proteins identified, 265 proteins were present in all samples. A final set of 181 proteins was used for the analysis after excluding immunoglobulin proteins and proteins without gene name annotations.

TABLE 14
Clinical characteristics. Recipient characteristics
at the time of transplant unless otherwise specified.

| | | | | Multivariate |
|---|---|---|---|---|
| | Died | Survived | p-value | p-value |
| N | 22 | 66 | | |

Patient characteristics

| | | | | | | |
|---|---|---|---|---|---|---|
| Age (mean (SD)) | 57.48 | (12.63) | 56.28 | (11.91) | 0.69 | 0.389 |
| BMI (mean (SD)) | 26.95 | (5.43) | 25.36 | (4.26) | 0.162 | 0.199 |
| Blood Type (%) | | | | | 0.054 | |
| A | 13 | (59.1) | 21 | (31.8) | | 0.881 |
| AB | 3 | (13.6) | 5 | (7.6) | | 0.51 |
| B | 1 | (4.5) | 12 | (18.2) | | 0.200 |
| O | 5 | (22.7) | 28 | (42.4) | | 0.062 |
| Donor age (mean (SD)) | 43.36 | (14.74) | 39.47 | (13.20) | 0.248 | 0.355 |
| Sex = F (%) | 10 | (45.5) | 17 | (25.8) | 0.142 | 0.21 |
| History of tobacco use = Y (%) | 6 | (27.3) | 25 | (37.9) | 0.519 | 0.885 |
| Diabetes = Y (%) | 11 | (50.0) | 18 | (27.3) | 0.089 | 0.383 |
| Cohort (%) | | | | | 0.115 | |
| Cedar-Sinai | 14 | (63.6) | 29 | (43.9) | | 0.55 |
| Columbia | 1 | (4.5) | 15 | (22.7) | | 0.27 |
| Pitié-Salpêtrière | 7 | (31.8) | 22 | (33.3) | | 0.99 |

Cardiomyopathy

| | | | | | | |
|---|---|---|---|---|---|---|
| Ischemic = Y (%) | 8 | (36.4) | 24 | (36.4) | 1 | 0.268 |
| Non-Ischemic (%) | | | | | 0.271 | |
| Adriamycin | 1 | (4.5) | 0 | (0.0) | | 1 |
| Amyloid | 0 | (0.0) | 2 | (3.0) | | 1 |
| Chagas | 0 | (0.0) | 1 | (1.5) | | — |
| Congenital | 1 | (4.5) | 0 | (0.0) | | 1 |
| Hypertrophic cardiomyopathy | 0 | (0.0) | 1 | (1.5) | | — |
| Idiopathic | 11 | (50.0) | 36 | (54.5) | | — |
| Myocarditis | 0 | (0.0) | 1 | (1.5) | | 1 |
| Valvular heart disease | 1 | (4.5) | 0 | (0.0) | | — |
| Viral | 0 | (0.0) | 1 | (1.5) | | — |

Transplant factors

| | | | | | | |
|---|---|---|---|---|---|---|
| PGD = Y (%) | 20 | (90.9) | 22 | (33.3) | <0.001 | 0.019 |
| Ischemic Time (min (SD)) | 154.45 | (61.18) | 165.19 | (57.73) | 0.459 | 0.048 |
| Ventricular Assist Device = Y (%) | 5 | (22.7) | 16 | (24.2) | 1 | 0.953 |

Hemodynamics

| | | | | | | |
|---|---|---|---|---|---|---|
| PA Diastolic (mean (SD)) mmHg | 20.05 | (8.08) | 20.74 | (6.98) | 0.7 | 0.092 |
| PA Systolic (mean (SD)) mmHg | 45.93 | (15.03) | 43.49 | (13.36) | 0.475 | 0.941 |
| PA Mean (mean (SD)) mmHg | 31.78 | (8.39) | 29.74 | (8.77) | 0.341 | 0.687 |
| CVP (mean (SD)) mmHg | 10.56 | (4.95) | 9.44 | (5.30) | 0.387 | 0.774 |
| PCWP (mean (SD)) mmHg | 21.21 | (8.13) | 19.52 | (8.34) | 0.408 | 0.200 |

Lab values

| | | | | | | |
|---|---|---|---|---|---|---|
| Creatinine (mean (SD)) mg/dL | 1.32 | (0.49) | 1.30 | (0.98) | 0.942 | 0.160 |
| INR (mean (SD)) | 1.73 | (0.80) | 1.50 | (0.55) | 0.135 | 0.063 |
| TBili (mean (SD)) mg/dL | 0.83 | (0.47) | 0.87 | (0.50) | 0.744 | 0.102 |
| Sodium (mean (SD)) mEq/L | 138.16 | (4.03) | 136.90 | (5.06) | 0.294 | 0.489 |

Medications

| | | | | | | |
|---|---|---|---|---|---|---|
| Antiarrhythmic Use = Y (%) | 15 | (68.2) | 32 | (48.5) | 0.175 | 0.200 |
| Beta Blocker = Y (%) | 15 | (68.2) | 39 | (59.1) | 0.613 | 0.143 |
| Inotrope = Y (%) | 7 | (31.8) | 37 | (56.1) | 0.085 | 0.176 |

Composite scores

| | | | | | | |
|---|---|---|---|---|---|---|
| CVP/PCWP (mean (SD)) | 0.54 | (0.27) | 0.51 | (0.27) | | |
| MELD-XI (mean (SD)) | 7.19 | (4.77) | 6.89 | (4.27) | 0.788 | 0.116 |

TABLE 15
Baseline clinical characteristics. Recipient characteristics at the time of transplant unless otherwise specified.

| | | | | | | Pitié | | |
|---|---|---|---|---|---|---|---|---|
| | | Cedar-Sinai | | Columbia | | Salpêtrière | | p-value |
| | N | 43 | | 16 | | 29 | | |
| Patient characteristics | Age (mean (SD)) | 57.95 | (12.76) | 56.50 | (10.28) | 54.60 | (11.91) | 0.517 |
| | BMI (mean (SD)) | 25.49 | (4.96) | 28.10 | (3.58) | 24.86 | (4.22) | 0.065 |
| | Blood Type (%) | | | | | | | 0.687 |
| | A | 17 | (39.5) | 6 | (37.5) | 11 | (37.9) | |
| | AB | 4 | (9.3) | 3 | (18.8) | 1 | (3.4) | |
| | B | 5 | (11.6) | 2 | (12.5) | 6 | (20.7) | |
| | O | 17 | (39.5) | 5 | (31.2) | 11 | (37.9) | |
| | Donor Age (mean (SD)) | 36.49 | (12.21) | 38.50 | (12.18) | 47.38 | (14.05) | 0.002 |
| | Sex = F (%) | 15 | (34.9) | 2 | (12.5) | 10 | (34.5) | 0.219 |
| | History of Tobacco Use = Y (%) | 2 | (4.7) | 11 | (68.8) | 18 | (62.1) | <0.001 |
| | Diabetes = Y (%) | 12 | (27.9) | 7 | (43.8) | 10 | (34.5) | 0.504 |
| | Survived = Y (%) | 29 | (67.4) | 15 | (93.8) | 22 | (75.9) | 0.115 |
| Cardiomyopathy | Ischemic = Y (%) | 12 | (27.9) | 8 | (50.0) | 12 | (41.4) | 0.231 |
| | Non-Ischemic (%) | | | | | | | 0.651 |
| | Adriamycin | 12 | (27.9) | 8 | (50.0) | 12 | (41.4) | |
| | Amyloid | 1 | (2.3) | 0 | (0.0) | 0 | (0.0) | |
| | Chagas | 2 | (4.7) | 0 | (0.0) | 0 | (0.0) | |
| | Congenital | 0 | (0.0) | 1 | (6.2) | 0 | (0.0) | |
| | Hypertrophic cardiomyopathy | 1 | (2.3) | 0 | (0.0) | 0 | (0.0) | |
| | Idiopathic | 1 | (2.3) | 0 | (0.0) | 0 | (0.0) | |
| | Myocarditis | 23 | (53.5) | 7 | (43.8) | 17 | (58.6) | |
| Transplant factors | Valvular Heart Disease | 1 | (2.3) | 0 | (0.0) | 0 | (0.0) | |
| | Viral | 1 | (2.3) | 0 | (0.0) | 0 | (0.0) | 0.927 |
| | PGD = Y (%) | 21 | (48.8) | 8 | (50.0) | 13 | (44.8) | |
| | Ischemic Time (minutes (SD)) | 148.33 | (65.49) | 178.36 | (44.66) | 174.79 | (50.02) | 0.081 |
| | Ventricular Assist Device = Y (%) | 8 | (18.6) | 11 | (68.8) | 2 | (6.9) | <0.001 |
| Hemodynamics | PA Diastolic (mean (SD)) mmHg | 19.85 | (6.35) | 15.62 | (7.72) | 24.35 | (6.37) | <0.001 |
| | PA Systolic (mean (SD)) mmHg | 41.37 | (12.08) | 36.81 | (12.59) | 52.17 | (13.21) | <0.001 |
| | PA Mean (mean (SD)) mmHg | 29.32 | (6.93) | 24.00 | (9.14) | 35.07 | (8.31) | <0.001 |
| | CVP (mean (SD)) mmHg | 10.54 | (5.27) | 7.00 | (5.79) | 10.00 | (4.39) | 0.062 |
| | PCWP (mean (SD)) mmHg | 18.99 | (7.16) | 15.38 | (10.29) | 23.87 | (7.06) | 0.002 |
| Lab values | Creatinine (mean (SD)) mg/dL | 1.41 | (1.21) | 1.28 | (0.32) | 1.18 | (0.32) | 0.54 |
| | INR (mean (SD)) | 1.47 | (0.56) | 1.71 | (0.73) | 1.60 | (0.66) | 0.387 |
| | TBili (mean (SD)) mg/dL | 0.75 | (0.29) | 0.53 | (0.31) | 1.21 | (0.60) | <0.001 |
| | Sodium (mean (SD)) mEq/L | 136.46 | (4.16) | 140.31 | (5.16) | 136.62 | (5.07) | 0.016 |
| Medications | Antiarrhythmic Use = Y (%) | 27 | (62.8) | 7 | (43.8) | 13 | (44.8) | 0.225 |
| | Beta Blocker = Y (%) | 25 | (58.1) | 14 | (87.5) | 15 | (51.7) | 0.051 |
| | Inotrope = Y (%) | 23 | (53.5) | 5 | (31.2) | 16 | (55.2) | 0.25 |
| Composite Scores | CVP/PCWP (mean (SD)) | 0.57 | (0.29) | 0.52 | (0.29) | 0.44 | (0.21) | 0.107 |
| | MELD (mean (SD)) | 13.47 | (5.18) | 14.44 | (5.23) | 14.31 | (4.50) | 0.702 |
| | RADIAL Score (mean (SD)) | 2.51 | (1.03) | 2.19 | (1.28) | 2.38 | (1.27) | 0.626 |

Abbreviations: Primary Graft Dysfunction, PGD; Body Mass Index, BMI; Pulmonary Artery, PA; Central venous pressure, CVP; Pulmonary capillary wedge pressure, PCWP; International Normalized Ratio, INR; Total bilirubin, TBili; Model for End Stage Liver Disease Score, MELD

Prediction of post-transplant survival using pre-transplant clinical and protein markers:

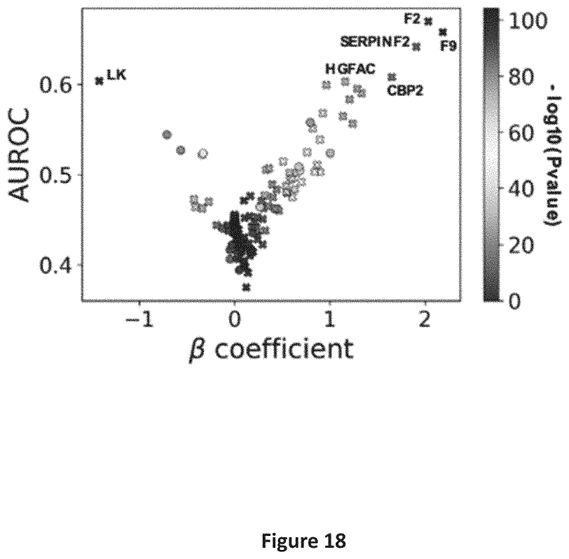

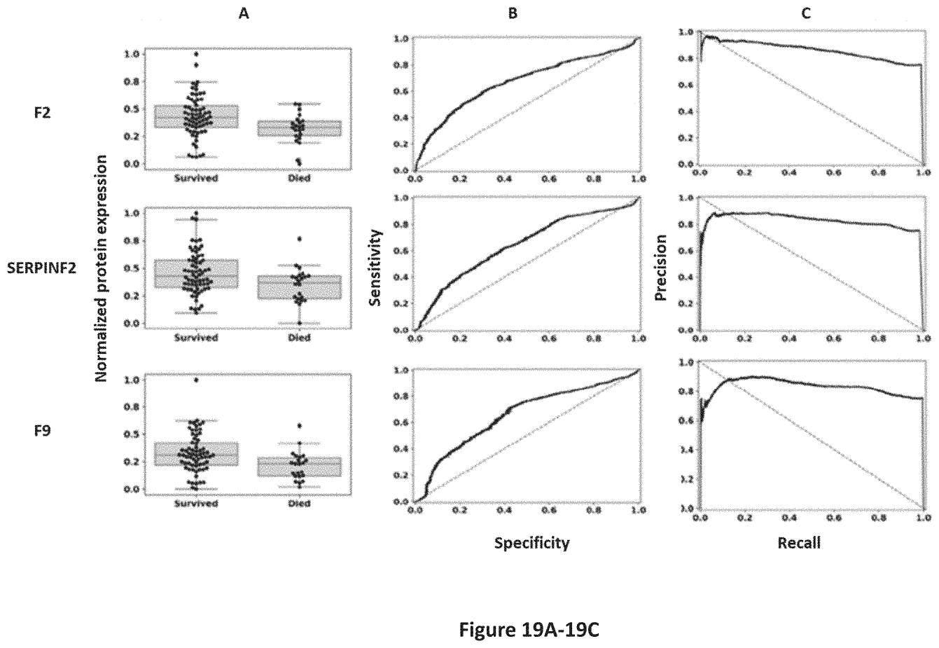

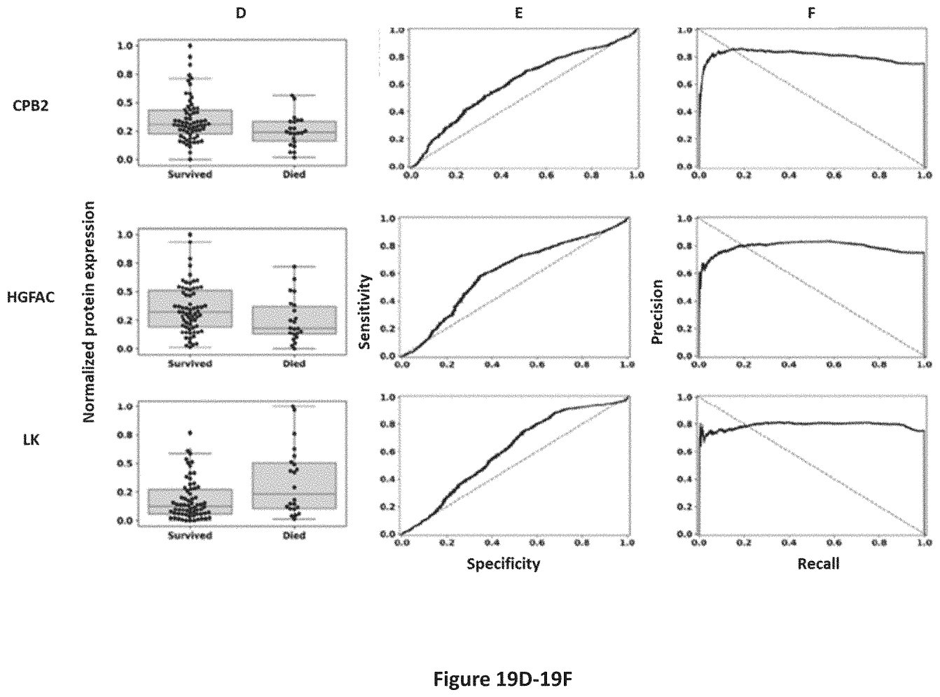

Monte Carlo Cross Validation (MCCV) and permutation analysis was employed to calculate the prediction interval and significance of each clinical and protein marker in predicting patient survival after heart transplant (FIG. 18). Eighteen clinical and protein markers were significantly predictive for patient survival after transplant (Table 16; AUROC>0.5, beta coefficient 95% confidence interval not containing the null association, and permutation beta coefficient interval containing the null association). After adjusting for patient site of origin, 11 clinical and protein markers remained significantly predictive of post-transplant survival (FIG. 18). Increased expression of prothrombin (F2), alpha 2-antiplasmin (SERPINF2), coagulation factor IX (F9), carboxypeptidase 2 (CPB2) and hepatocyte growth factor activator (HGFAC) and decreased expression of low molecular weight kininogen (LK) were found to be most predictive (AUROC>0.6) of patient survival (FIG. 19A-19F).

TABLE 16
Significant markers of post-transplant survival. Bold values significantly
predicted post-transplant survival after adjustment for patient site-of-origin. The positive control,
primary graft dysfunction (PGD)

| | AUROC | | AUROC | Beta | | Beta |
|---|---|---|---|---|---|---|
| | 2.5% | AUROC | 97.5% | 2.50% | Beta | 97.5% |
| F9 | 0.634 | 0.658 | 0.685 | 1.261 | 2.188 | 3.443 |
| F2 | 0.649 | 0.67 | 0.684 | 1.24 | 2.036 | 2.758 |
| SERPINF2 | 0.621 | 0.642 | 0.663 | 0.975 | 1.909 | 2.662 |
| CPB2 | 0.59 | 0.608 | 0.631 | 0.8 | 1.651 | 2.782 |
| ITIH2 | 0.574 | 0.59 | 0.611 | 0.663 | 1.334 | 1.998 |
| FBLN1 | 0.569 | 0.595 | 0.618 | 0.443 | 1.288 | 2.432 |
| CLEC3B | 0.536 | 0.556 | 0.579 | 0.413 | 1.24 | 2.057 |
| HPR | 0.559 | 0.583 | 0.604 | 0.444 | 1.207 | 2.297 |
| HGFAC | 0.584 | 0.603 | 0.628 | 0.335 | 1.162 | 2.168 |
| CD5L | 0.543 | 0.564 | 0.583 | 0.265 | 1.138 | 2.018 |
| KRT10 | 0.579 | 0.599 | 0.618 | 0.075 | 0.963 | 1.994 |
| FCN2 | 0.518 | 0.539 | 0.563 | 0.053 | 0.894 | 1.753 |
| Inotrope | 0.536 | 0.558 | 0.578 | 0.428 | 0.794 | 1.248 |
| Therapy | | | | | | |
| PF4 | 0.505 | 0.525 | 0.543 | 0.114 | 0.762 | 1.769 |
| Diabetes | 0.508 | 0.527 | 0.555 | −0.956 | −0.566 | −0.099 |
| Blood type A | 0.526 | 0.544 | 0.57 | −1.016 | −0.712 | −0.298 |
| LK | 0.585 | 0.604 | 0.628 | −2.272 | −1.421 | −0.445 |
| PGD | 0.706 | 0.723 | 0.744 | −2.513 | −2.055 | −1.726 |

Comparative analysis to determine predictive profiles between near term (<1 year) and long term (>1 year) survival, diminished the number of mortality events and thus the power of the analysis as 7 of 22 deaths occurred after one year. Among the markers, SERPINF2, F9, and LK remained significant predictors while F2, CPB2 and HGFAC were no longer predictive (Table 17). This demonstrated that there was some attenuation of prediction performance in several of the proteins when focusing on 1 year survival, though the predictive metrics of those proteins that remained significant were unchanged.

TABLE 17
Comparison of significantly predictive proteins
between survival prediction schemes.

| | Survival | | Survival | Survival (all-time) |
|---|---|---|---|---|
| | (all-time) | | (1-year) | with PGD covariate |
| F2 | 0.67 | [0.649, 0.684] | — | — |
| SERPIN F2 | 0.642 | [0.621, 0.663] | 0.651 [0.632, 0.678] | 0.826 [0.812, 0.845] |
| F9 | 0.658 | [0.634, 0.685] | 0.675 [0.658, 0.697] | 0.842 [0.825, 0.857] |
| CPB2 | 0.608 | [0.590, 0.631] | — | 0.832 [0.818, 0.843] |
| HGFAC | 0.603 | [0.584, 0.628] | — | 0.793 [0.776, 0.808] |
| LK | 0.604 | [0.585, 0.628] | 0.678 [0.654, 0.707] | 0.804 [0.786, 0.820] |

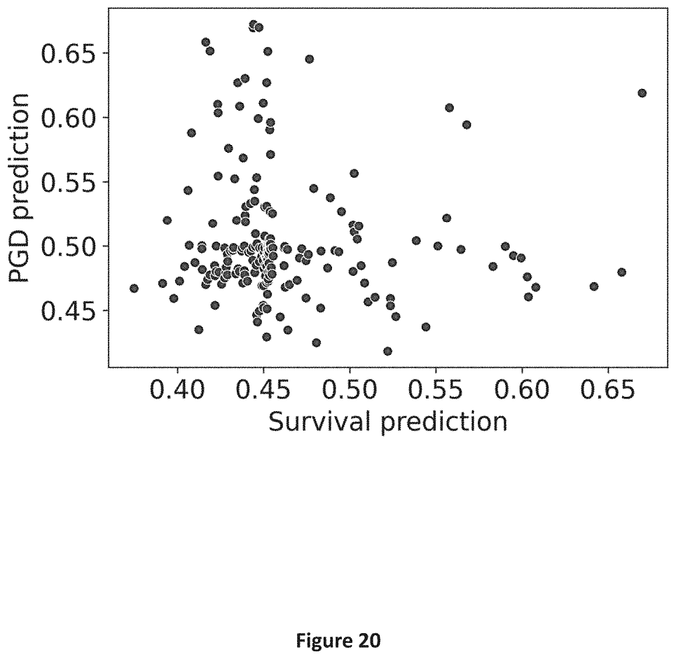

In a secondary control analysis, PGD, known to be associated with mortality was found to be a predictive clinical marker (AUROC: 0.723 [0.706, 0.744], Beta coeffcient: −2.06 [−2.514, −1.726]) (Table 16). Though this analysis is agnostic to the cause of death, the prevalence of PGD in this cohort raises the question of whether the predictive performance of the proteins is in some way linked to PGD. To ascertain this, the analysis was performed accounting for PGD status as a covariate, where all predictive proteins had higher performance (AUROC>0.71) when accounting for PGD, demonstrating that prediction was not dependent on PGD status (Table 17). Comparison of the predictive performance of proteins for survival to PGD did not reveal a statistically significant association (Spearman rho coeffcient=0.074, p-value=0.3, FIG. 20).

4. Post-Transplant Survival Differential Signature.

Biological pathways associated prior to heart transplant to elucidate putative mechanisms contributing to patient survival were investigated. There were 262 proteins expressed in all patients including immunoglobulins to compute a differential protein signature. Immunoglobulins were not significantly different, on average, from non-immunoglobulins across patients (Mann Whitney p-value=0.264). Gene set enrichment analysis was utilized on differential protein expression and pathways and functions (FDR<0.2) were found to be enriched for post-transplant survival (Tables 18 and 19). Enriched pathways associated with survival included platelet activation and the coagulation cascade. Of the predictive proteins with AUROC>0.6, F2, F9, CPB2, SERPINF2 and LK were all components within the kallikrein-kinin pathway.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

TABLE 18
Significantly enriched pathways for post-transplant patient survival. Significance
evaluated by phenotype permutation False Discovery Rate < 0.2. Sorted by FDR

| | Normalized enrichment | False discovery |
|---|---|---|
| | score | rate |
| Response to elevated platelet cytosolic Ca2+_Homo sapiens_R-HSA-76005 | 4.156 | <0.001 |
| Extracellular matrix organization_Homo sapiens_R-HSA-1474244 | 4.163 | <0.001 |
| Platelet degranulation_Homo sapiens_R-HSA-114608 | 4.156 | <0.001 |
| Metabolism of proteins_Homo sapiens_R-HSA-392499 | −3.418 | 0.116 |
| Platelet activation, signaling and aggregation_Homo sapiens_R-HSA-76002 | 3.783 | 0.138 |
| Signal Transduction_Homo sapiens_R-HSA-162582 | 3.546 | 0.189 |

TABLE 19
Significantly enriched pathways for post-transplant patient survival. by gene set permutation.

| | Normalized Enrichment | False Discovery |
|---|---|---|
| | Score | Rate |
| Complement and Coagulation Cascades WP558 | 1.575 | 0.065 |
| Metabolism of proteins_Homo sapiens_R-HSA-392499 | 1.684 | 0.108 |
| calcium ion binding involved in regulation of cytosolic calcium ion concentration (GO:0099510) | 1.511 | 0.122 |
| Formation of Fibrin Clot (Clotting Cascade)_Homo sapiens_R-HSA-140877 | 1.731 | 0.14 |
| calcium ion sensor activity (GO:0061891) | 1.521 | 0.153 |
| sarcoplasmic reticulum lumen (GO:0033018) | 1.419 | 0.155 |
| cortical endoplasmic reticulum lumen (GO:0099021) | 1.44 | 0.161 |
| serine-type endopeptidase activity (GO:0004252) | 1.55 | 0.184 |

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A method for identifying risk of primary graft dysfunction (PGD) of a subject comprising:
Collecting a sample of the subject;
measuring a level of a PGD marker from the sample, wherein the PGD marker comprises plasma kallikrein (KLKB1);
providing a PGD risk value that is quantified based on the level of the PGD marker using an adaptive Monte Carlo cross-validation (MCCV) model; and
identifying the risk of PGD based on the PGD risk value.

2. The method of claim 1, further comprising assessing an effect of a therapy on the heart transplant by estimating the PGD risk value of the subject, wherein the subject receives the therapy before or after the assessing.

3. The method of claim 1, further comprising identifying a clinical variable of the subject, wherein the clinical variable comprises a medical history of the subject.

4. The method of claim 3, wherein the medical history of the one subject comprises a pre-transplant inotrope therapy.

5. The method of claim 1, further comprising measuring a level of an additional marker from the sample, wherein the additional marker is selected from the group consisting of proteins peroxiredoxin 2 (PRDX2), tropomyosin alpha-4 (TPM4), myeloperoxidase (MPO), PGLYRP2, DEFA1, DEFA1B, LDHB, F2, FCGBP, CAT, CFHR5, HIST1H4, GAPDH, LTF, ADIPOQ, HSPA5, and combinations thereof.

6. The method of claim 5, wherein the PGD risk value is quantified based on the level of the PGD marker and the additional marker.

7. The method of claim 1, further comprising providing the adaptive MCCV model with a training set for machine learning, wherein the adaptive MCCV model is a continuously evolving model based on the training set.

8. The method of claim 1, further comprising providing an additional therapy to the subject based on the PGD risk value.

9. The method of claim 8, wherein the additional therapy comprises KLKB1 activators, anti-inflammatory agents, or combinations thereof.

10. A system for identifying risk of primary graft dysfunction (PGD) of a subject comprising:
one or more processors; and
one or more computer-readable non-transitory storage media coupled to one or more of the processors and comprising instructions operable when executed by one or more of the processors to cause the system to:
collect a sample of the subject;
measure a level of a PGD marker from the sample, wherein the PGD marker comprises plasma kallikrein (KLKB1);
provide a PGD risk value that is quantified based on the level of the PGD marker using an adaptive Monte Carlo cross-validation (MCCV) model; and
identify the risk of PGD based on the PGD risk value.

11. The system of claim 10, wherein the processor is configured to assess an effect of a therapy on the heart transplant by estimating the PGD risk value of the subject, wherein the subject receives the therapy before or after the assessing.

12. The system of claim 10, wherein the processor is configured to identify a clinical variable of the subject, wherein the clinical variable comprises a medical history of the subject.

13. The system of claim 12, wherein the medical history of the one subject comprises a pre-transplant inotrope therapy.

14. The system of claim 10, wherein the processor is configured to measure a level of an additional marker from the sample, wherein the additional marker is selected from the group consisting of proteins peroxiredoxin 2 (PRDX2), tropomyosin alpha-4 (TPM4), myeloperoxidase (MPO), PGLYRP2, DEFA1, DEFA1B, LDHB, F2, FCGBP, CAT, CFHR5, HIST1H4, GAPDH, LTF, ADIPOQ, HSPA5, and combinations thereof.

15. The system of claim 14, wherein the PGD risk value is quantified based on the level of the PGD marker and the additional marker.

16. The system of claim 10, wherein the processor is configured to provide the adaptive MCCV model with a training set for machine learning, wherein the adaptive MCCV model is a continuously evolving model based on the training set.

17. The system of claim 10, the system is configured to provide an additional therapy to the subject based on the PGD risk value.

18. The system of claim 17, wherein the additional therapy comprises KLKB1 activators, anti-inflammatory agents, or combinations thereof.

19. A method for predicting post-transplant survival of a subject seeking an organ transplant comprising:
collecting a sample from the subject;
measuring in the sample, a level of a marker predictive of post-transplant survival;
providing a transplant risk value that is quantified based on the level of the marker using an adaptive Monte Carlo cross-validation (MCCV) model; and
predicting the likelihood of post-transplant survival based on the transplant risk value.

20. The method of claim 19, wherein predicting post-transplant survival identifies a risk of primary graft dysfunction (PGD).

21. The method of claim 19, wherein the marker predictive of post-transplant survival is at least one of prothrombin (F2), anti-plasmin (SERPINF2), Factor IX (F9), carboxypeptidase 2 (CPB2), HGF activator (HGFAC) and low molecular weight kininogen (LK).

22. The method of claim 21, wherein a level of F2, SERPINF2, F9, CPB2, or HGFAC outside a distribution of values in a survival cohort, or a level of LK outside a distribution of values in a survival cohort predicts post-transplant survival of the subject.

23. The method of claim 19, wherein the marker predictive of post-transplant survival is SERPINF2, F9, or LK, or a combination thereof.

24. The method of claim 19, further comprising providing the adaptive MCCV model with a training set for machine learning, wherein the adaptive MCCV model is a continuously evolving model based on the training set.

25. The method of claim 19, further comprising providing a therapy to the subject based on the transplant risk value, wherein the subject receives the therapy before or after the organ transplant.

26. The method of claim 19, further comprising identifying a clinical variable of the subject, wherein the clinical variable comprises a medical history of the subject.
